(19) United States
(12) Patent Application Publication
(10) Pub. No.: US 2020/0265955 A1
(43) Pub. Date: Aug. 20, 2020

(54) Identifying Valid Medical Data for Facilitating Accurate Medical Diagnosis

(71) Applicant: Babylon Partners Limited, London (GB)

(72) Inventors: Domenico CORAPI, London (GB); Myles BELL, London (GB); Ansis STRODAHS, London (GB); Benjamin HOMER, London (GB); Maurizio MORRIELLO, London (GB); Mohammad KHODADADI, London (GB)

(21) Appl. No.: 16/546,231

(22) Filed: Aug. 20, 2019

Related U.S. Application Data
(63) Continuation of application No. 16/275,901, filed on Feb. 14, 2019.

(51) Int. Cl. G16H 50/20; G16H 10/60; G16H 50/30; G06N 7/00; G06F 16/23

(52) U.S. Cl. CPC G16H 50/20; G16H 10/60; G06F 16/2365; G06N 7/005; G16H 50/30

(57) ABSTRACT

Methods for determining validity of medical information are provided. In one aspect, a computer-implemented method includes obtaining, from a stored clinical history, information relating to a set of items of medical data associated with a user, the information comprising an indication of whether the item of medical data is present, wherein an item of medical data comprises a symptom, risk factor, disease, physiological data, recommendation or behaviour. The method also includes obtaining the source of the information indicating whether the item of medical data is present. The method also includes determining the validity of the information and providing valid information, wherein the validity is determined from information indicating items of medical data which are permanently valid or from a time duration from when the information was reported in the stored clinical history. The method also includes applying a policy to prioritise information based on the source information.

### CROSS-REFERENCE TO RELATED APPLICATION

This application claims the benefit of priority under 35 U.S.C. § 120 as a continuation of U.S. patent application Ser. No. 16/275,901, filed on Feb. 14, 2019, the contents of which are hereby incorporated by reference in its entirety for all purposes.

### FIELD

Embodiments described herein relate to methods and systems for medical diagnosis. In particular, such methods and systems may determine a probability of a disease from information input by a user and from information retrieved from the user's clinical history. Further embodiments relate to methods and systems for determining validity of the information.

### BACKGROUND

Medical diagnosis may use knowledge of symptoms experienced by a patient, combined with information regarding risk factors, for example, to identify medical conditions (diseases). This may allow offering possible treatments to the patient.

In many cases, medical diagnosis is based on making a decision by considering the causal and probabilistic relationship between items of medical data such as risk factors, diseases, and symptoms. Medical models may be used to describe the interplay between items of the medical data. For example, a model that elegantly captures such causal relationships is based on the framework of probabilistic graphical models (PGM). Key to decision-making in such a system is the process of performing probabilistic inference on the PGM.

Such systems determine the likelihood of a set of diseases, based on available evidence. The available evidence is provided by a user. However, the evidence provided may be incomplete. With incomplete evidence, the likelihood of diseases may be difficult to predict and the accuracy of the diagnosis may be poor.

### BRIEF DESCRIPTION OF THE DRAWINGS

Systems and methods in accordance with non-limiting embodiments will now be described with reference to the accompanying figures in which:

FIG. 1 is a schematic illustration of an exemplary medical diagnosis system;

FIG. 2 is a schematic illustration of a medical diagnosis system in accordance with an embodiment;

FIG. 3(a) is a depiction of a probabilistic graphical model (PGM) used in a medical diagnosis system in accordance with an embodiment;

FIG. 3(b) illustrates an importance sampling method used in a medical diagnosis method in accordance with an embodiment;

FIG. 3(c) illustrates an example training process for a universal marginalizer (UM) used in a medical diagnosis system in accordance with an embodiment;

FIG. 4 is a schematic illustration of a medical diagnosis system in accordance with an embodiment;

FIG. 5 illustrates a method for medical diagnosis in accordance with an embodiment;

FIG. 6(a) is a schematic illustration of a system for determining the validity of medical information in accordance with an embodiment;

FIG. 6(b) is a schematic illustration of a system for determining the validity of medical information in accordance with an embodiment;

FIG. 6(c) shows a flow chart illustrating a method for determining the validity of medical information in accordance with an embodiment;

FIG. 6(d) shows a flow chart of a method for determining the validity of medical information in accordance with an embodiment;

FIG. 7 shows a medical diagnosis system according to an embodiment, comprising a concept reasoner;

FIG. 8(a) shows a method of processing the user input using a concept reasoner, which may be used in a method of medical diagnosis in accordance with an embodiment;

FIG. 8(b) is a flowchart depicting the first part of an example Algorithm 2 which may be used in the method of FIG. 8(a);

FIG. 8(c) is a flowchart depicting the second part of an example Algorithm 2 which may be used in the method of FIG. 8(a);

FIG. 9 is a schematic of a computing system which provides means capable of putting a method for medical diagnosis in accordance with an embodiment into effect.

### DETAILED DESCRIPTION

According to a first aspect of the invention, there is provided a computer-implemented method for medical diagnosis, the method comprising receiving an input from a user comprising at least one input symptom. The method also includes identifying the user. The method also includes determining the validity of information relating to one or more items of medical data from a set of stored information relating to medical data associated with the user. The method also includes providing the at least one input symptom, and valid information relating to the one or more items of medical data, as an input to a model, the model being configured to output a probability of the user having a disease. The method also includes outputting a diagnosis based on the probability of the user having a disease.

According to a second aspect of the invention, there is provided a medical diagnosis system comprising a user interface configured to receive an input from a user comprising at least one input symptom. The system also includes a processor configured to identify the user. The processor is also configured to determine the validity of information relating to one or more items of medical data from a set of stored information relating to medical data associated with the user. The processor is also configured to provide the at least one input symptom, and the valid information relating to the one or more items of medical data, as an input to a model, the model being configured to output a probability of the user having a disease. The system also includes a display device configured to display a diagnosis based on the probability of the user having a disease.

The disclosed system provides an improvement to computer functionality by allowing computer performance of a function not previously performed by a computer. Specifically, the disclosed system provides a solution to the technical problem that when a diverse data set (e.g., the patient's clinical history) comprising information relating to items of medical data is used to perform diagnosis, conflicting, incomplete, irrelevant, or erroneous data points may be inputted to the diagnosis system. For example, it is likely that the data set may comprise data points entered by non-expert users (the patients), data points entered by human doctors, and data from medical databases, with multiple conflicting data points entered at different times. This may prevent the system from performing a sufficiently accurate diagnosis. The disclosed system solves this technical problem by processing the stored data set to identify a subset of valid information such that an accurate diagnosis can be obtained.

The disclosed system further addresses a technical problem tied to computer technology and arising in the realm of computer networks, namely the technical problem of inefficient use of computational resources. The disclosed system solves this technical problem by storing a set of information relating to items of medical data associated with a user, and, when an input symptom is received associated with a user, determining the validity of the stored information. In this way, it is not required to continuously update the validity of each piece of information; rather, this is determined when required. Moreover, as methods of determining the validity of the stored information are improved, it is not required to update the validity of the information. Less computation is therefore required.

In an embodiment, an item of medical data is a symptom, risk factor, or disease. In an embodiment, an item of medical data is a symptom, risk factor, disease, physiological data, recommendation, or behaviour. In a further embodiment, the information comprises information indicating that the symptom, risk factor, disease, physiological data, recommendation, or behaviour is present. In a further embodiment, the stored information comprises information indicating that the symptom, risk factor, disease, physiological data, recommendation, or behaviour is present or indicating that it is absent.

In another embodiment, determining the validity of the information is performed based on a property of the information. In a further embodiment, the property is a time duration from when the information was reported. In another embodiment, determining the validity of the information is performed based on a property of the item of medical data. In a further embodiment, the property is permanence.

In another embodiment, the input to the medical model is obtained by combining the input from the user with the valid information, according to a pre-defined priority based on source information indicating the source of the information relating to the item of medical data.

In another embodiment, if the source information indicates that the source is a human doctor, the information has priority over information having a different source.

In another embodiment, the input to the medical model is obtained by combining the input from the user with the valid information according to a pre-defined priority based on the time the information was reported.

In another embodiment, determining the validity comprises identifying any conflicting information, and requesting the user to confirm whether some or all of the conflicting entries are valid.

In an embodiment, some or all of the valid information is confirmed with the user before being input to the model.

In another embodiment, determining the validity comprises comparing a reference time duration to the time duration from when the information was reported, and determining that the information is valid if the reference time duration is greater than the time duration from when the information was reported.

In another embodiment, the stored information comprises information indicating that the item of medical data is present, and determining the validity comprises comparing the item of medical data to a list of items of medical data which are permanently valid, and determining that the information indicating that the item of medical data is present is valid if the item of medical data corresponds to an entry on the list.

In another embodiment, determining the validity comprises comparing a reference time duration to a time duration from when the information was reported, and determining that the information is valid if the reference time duration is greater than the time duration from when the information was reported, wherein for one or more of the items of medical data, there is a first reference time duration which is used when the information indicates that the item is present, and a second reference time duration which is used when the information indicates that the item is absent.

In an embodiment, said model comprises a probabilistic graphical model containing probability distributions and relationships between symptoms and diseases, and an inference engine configured to perform Bayesian inference on said probabilistic graphical model, and wherein determining the probability that the user has a disease comprises performing approximate inference on the probabilistic graphical model to obtain a prediction of the probability that the user has a disease.

In another embodiment, the method further comprises obtaining a set of symptoms, risk factors and/or diseases to be used in the probabilistic graphical model. The method also includes requesting the stored information relating to the symptoms, risk factors and/or diseases to be used in the model associated with the user. The method also includes determining the validity of the requested information.

In another embodiment, inference is performed using a discriminative model, wherein the discriminative model has been pre-trained to approximate the probabilistic graphical model, the discriminative model being trained using samples generated from said probabilistic graphical model, wherein some of the data of the samples has been masked to allow the discriminative model to produce data which is robust to the user providing incomplete information about their symptoms, and wherein determining the probability that the user has a disease comprises deriving estimates of the probabilities that the user has that disease from the discriminative model, inputting these estimates to the inference engine and performing approximate inference on the probabilistic graphical model to obtain a prediction of the probability that the user has that disease.

In another embodiment, the method further comprises obtaining a set of symptoms, risk factors, and/or diseases to be used in the model. The method also includes checking if a symptom, risk factor, or disease from the set of stored information associated with the user has a subsumption relationship with a candidate symptom, risk factor, and/or disease to be used in the model.

In another embodiment, the method further comprises checking if a symptom, risk factor, or disease from the set of stored information associated with the user has a subsumption relationship with a candidate symptom, risk factor, and/or disease for which information used to determine validity is stored.

According to a third aspect of the invention, there is provided a computer-implemented method for determining validity of medical information, the method comprising obtaining information relating to a set of items of medical data associated with a user. The method also includes determining validity of the information. The method also includes providing validated information.

According to a fourth aspect of the invention, there is provided a system for determining validity of medical information, the system comprising an input configured to receive information relating to a set of items of medical data associated with a user. The system also includes a processor configured to determine the validity of the information. The system also includes an output configured to provide the valid information.

According to a fifth aspect of the invention, there is provided a carrier medium comprising computer-readable code configured to cause a computer to perform the above described methods.

According to a sixth aspect of the invention, there is provided a computer-implemented method for determining validity of medical information, the method including obtaining, from a stored clinical history, information relating to a set of items of medical data associated with a user, the information comprising an indication of whether the item of medical data is present, wherein an item of medical data comprises a symptom, risk factor, disease, physiological data, recommendation or behaviour. The method also includes obtaining, from the stored clinical history, the source of the information indicating whether the item of medical data is present. The method also includes determining the validity of the information and providing valid information, wherein the validity is determined from information indicating items of medical data which are permanently valid or from a time duration from when the information was reported in the stored clinical history. The method also includes applying a policy to prioritise information based on the source information.

According to a seventh aspect of the invention, there is provided a computer-implemented method for determining validity of medical information, the method including obtaining, from a stored clinical history, information relating to a set of items of medical data associated with a user, the information comprising an indication of whether the item of medical data is present, wherein an item of medical data comprises a symptom, risk factor, disease, physiological data, recommendation or behaviour. The method also includes obtaining, from the stored clinical history, the source of the information indicating whether the item of medical data is present. The method also includes determining the validity of the information and providing valid information, wherein the validity is determined from information indicating items of medical data which are permanently valid or from a time duration from when the information was reported in the stored clinical history. The method also includes applying a policy to exclude valid information based on the source information.

The methods are computer-implemented methods. Since some methods in accordance with embodiments can be implemented by software, some embodiments encompass computer code provided to a general purpose computer on any suitable carrier medium. The carrier medium can comprise any storage medium such as a floppy disk, a CD ROM, a magnetic device or a programmable memory device, or any transient medium such as any signal, e.g., an electrical, optical, or microwave signal. The carrier medium may comprise a non-transitory computer-readable storage medium.

The above described systems and methods determine the likelihood of a set of diseases, based on available evidence, and provide a diagnosis. The available evidence is generated from information provided by a user combined with information relating to the user that is collected from previous interactions (e.g., past diagnoses) and stored in the user's clinical history. Further information may also be requested from the user to provide a more accurate diagnosis. Several requests may be made. Evidence may comprise a list of items of medical data which are present. The items of medical data may be risk factors, diseases, symptoms, physiological data, recommendations, or behaviours. Evidence may further comprise any other information that can be used to provide a diagnosis.

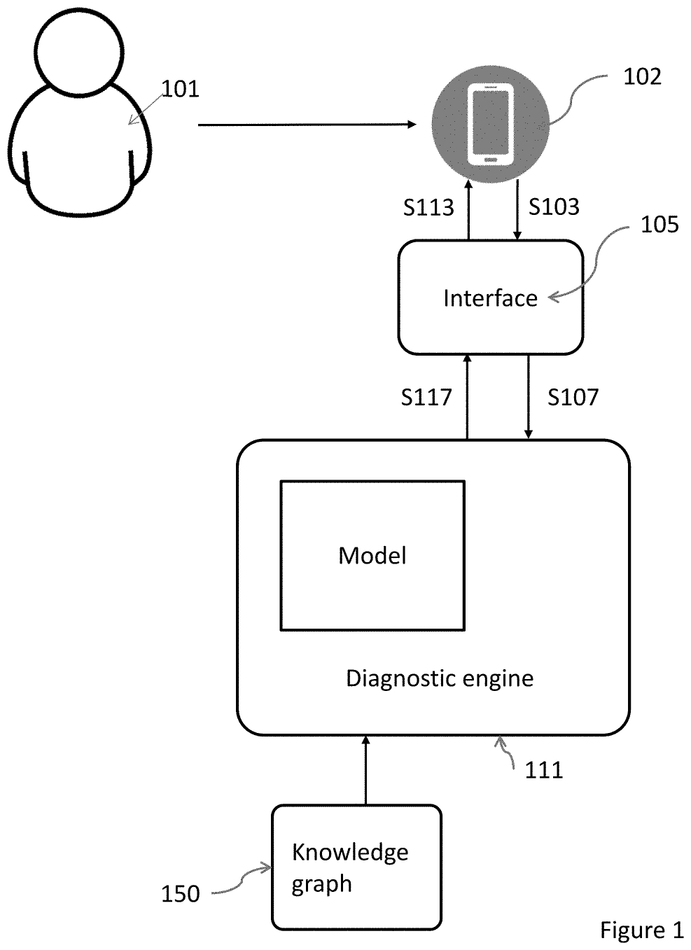

FIG. 1 illustrates an exemplary medical diagnosis system. A patient 101 communicates with the system via a mobile phone 102. However, any device that is capable of communicating information over a computer network, for example, a laptop, tablet computer, information point, fixed computer, etc., could be used. The device is also referred to as a user terminal. The user terminal is capable of conveying information to the patient in any suitable form. Such forms may include images and text by means of a display, or sounds and speech by means of a loudspeaker. The user terminal may also receive inputs from the user in different forms, for example by the user entering text, selecting from text options displayed on the terminal, or speaking into the device, for example.

The mobile phone 102 will communicate with interface 105, and transmit the text information corresponding to the user input to the interface 105 in S103. Interface 105 has two primary functions; the first function is to receive the words input by the user. In step S107, this information is passed on to the diagnosis engine 111. The second function is to take the output of the diagnosis engine 111 and to send this back to the user's mobile phone 102 in steps S117 and S113.

The patient 101 inputs their symptoms in step S103 via interface 105. The patient may also input other items of medical data which are present, such as their risk factors (for example, whether they are a smoker, etc.) and/or known diseases (for example, they have diabetes or asthma). The interface may be adapted to ask the patient 101 specific questions. Alternately, the patient may simply enter free text.

The diagnostic engine 111 receives the input text information from the interface 105 in step S107. The diagnostic engine then calls an NLP module (not shown) that applies NLP techniques to the input text information to extract concepts. This NLP module converts the natural text into concepts. Concepts will be described in detail below.

For example, in this step, following receipt of an input phrase, the diagnostic engine 111 calls an NLP dependency parser which extracts one or more symptoms which are present in the phrase, and outputs the concept(s) corresponding to the symptom(s). Various publicly available parsers can be used. In this example, the output of the NLP module therefore comprises a set of concepts (in this case representing symptoms) that are present in the input phrase. The subset of these concepts corresponding to the items of medical data used in the model is then extracted.

The evidence input into the model may comprise the presence of items of medical data, such as symptoms, diseases, risk factors, physiological data, recommendations and/or behaviours, that are identified as such by the patient in the input. It may further comprise the absence of items of medical data, such as symptoms, diseases, risk factors, physiological data, recommendations, and/or behaviours, identified as such by the patient in the input. In a simple example, the evidence comprises a set of symptoms which are present. For items of medical data, where the patient has not provided information, these will be assumed to be unknown.

Next, this evidence is passed in step S107 to the diagnosis engine 111. In the example description below, the evidence relates to symptoms, diseases, and/or risk factors. However, it will be understood that the input evidence may include information relating to any items of medical data, including additional items of medical data such as described previously. The diagnosis engine 111 is configured to compute probabilities that diseases are present based on evidence (e.g., the presence of one or more symptoms, diseases, and/or risk factors) provided, and derive a diagnosis from these probabilities. The diagnosis engine 111 comprises a model of medicine, which encompasses human knowledge of medicine, and an inference engine, which quantifies the likelihood of a disease being present, in view of the reported evidence (e.g., a list of symptoms, diseases, and/or risk factors which are identified as present) and the model of medicine, for example. The ‘model of medicine’ may be encoded in several ways, for example, it may be a PGM.

The diagnostic engine 111 may then transmit back information in step S117 concerning the “likelihood” of a disease, and generate a diagnosis, given the evidence supplied by the patient 101. The interface 105 can supply this information back to the mobile phone 102 of the patient in step S113. The information may alternatively be outputted to a different device, for example a computer operated by a doctor. The information is then displayed on a display device. For example, the mobile phone 102 of the patient may be configured to display a diagnosis based on the probability of the user having a disease.

The diagnostic engine 111 may be connected to a knowledge graph 150, the knowledge graph being a large structured medical knowledge base. The knowledge graph can be thought of as a repository of human knowledge on modern medicine encoded in a manner that can be understood by machines. The knowledge graph may keep track of the meaning behind medical terminology across different medical systems and different languages. The diagnostic engine 111 uses the medical knowledge encoded in the knowledge graph and calls the NLP module described above to turn the words input by the user into a form that can be understood by the model, i.e., concepts. Each item of medical data such as a symptom, disease, or risk factor, for example, corresponds to a concept.

The knowledge graph comprises a set of simple concepts (such as “headache”), encoded using medical information. Each simple concept comprises a label (e.g., “headache”) and an identifier (e.g., an IRI, discussed below). The knowledge graph may be understood to comprise simple concepts, and the relationships between them. Complex concepts may be constructed from two or more simple concepts from the knowledge graph, but are not themselves stored in the knowledge graph. Complex concepts such as “severe headache” may be constructed from a simple concept and one or more modifiers, for example.

The relationships may be links between the simple concepts. For example, a simple concept may be “pain.” This simple concept may be a “parent” concept for several “child” simple concepts such as “headache,” etc. These “child” concepts may also be “parent” concepts for further “child” concepts such as “frontal headache.” The child concept “frontal headache” is thus a simple concept that exists in the knowledge graph, and comprises a label and an identifier:

```
{
  “label”: “Frontal headache”,
  “iri”: “https://bbl.health/hFEPy0dO2k”
}
```

“Severe headache” in this example is a complex concept which does not exist in the knowledge graph, however. The complex concept “severe headache” may be represented as follows:

```
{
  “baseConcept”: {
    “label”: “Headache”,
    “iri”: “https://bbl.health/eD42RdeKVT”
  },
  “modifiers”: [
    {
      “type”: {
        “label”: “Severity”,
        “iri”: “https://bbl.health/PvSutVtoiC”
      },
      “value”: {
        “label”: “Severe”,
        “iri”: “https://bbl.health/m7MbriuuZ8”
      }
    }
  ]
}
```

The complex concept “severe headache” does not exist in the knowledge base, but it can be created by putting three simple concepts, i.e., three identifiers, from the knowledge base together. The identifiers may identify simple concepts that exist in the knowledge base. In the example of “severe headache” above, the modifier “Severity” is also a simple concept, having a “value” of “severe” in this case. “Severe” is also a simple concept.

Frontal headache in this example is a simple concept which exists in the knowledge base itself. It may also be represented as a complex concept, however.

Concepts may also include unary concepts, e.g., Not Headache. In this example, the “Not” is a special operator. The operator “Not” does not exist in the knowledge base, but it is part of the algebra that is used to define the semantics of the language.

Each simple concept comprises a corresponding identifier that uniquely identifies it. The identifier may be compatible with a standard protocol known as the Internationalized Resource Identifier (IRI), for example. An IRI may be understood as a string of characters that identifies a resource. An IRI may be a link to an internet-based resource. For example, the symptom headache may correspond to a concept comprising the label “headache” and an identifier that is an IRI, and the IRI is linked by, e.g., “http://health/test123.” The knowledge base stores simple concepts, each comprising a label and an identifier.

As described above, where the user input comprises free text, the diagnostic engine 111 calls one or more services to extract concepts from the text. For example, a natural language parser which extracts the concepts which are present in the text and corresponding to one or more simple concepts in the knowledge base may be used. For example, a word2vec model may produce word embeddings, which are then mapped to concepts in the knowledge base. The diagnostic engine may then extract the concepts which correspond to concepts in the model used by the diagnostic engine. In other cases, the mapping may be pre-coded (for example, given a question presented to the user, each answer corresponds to a concept used in the model).

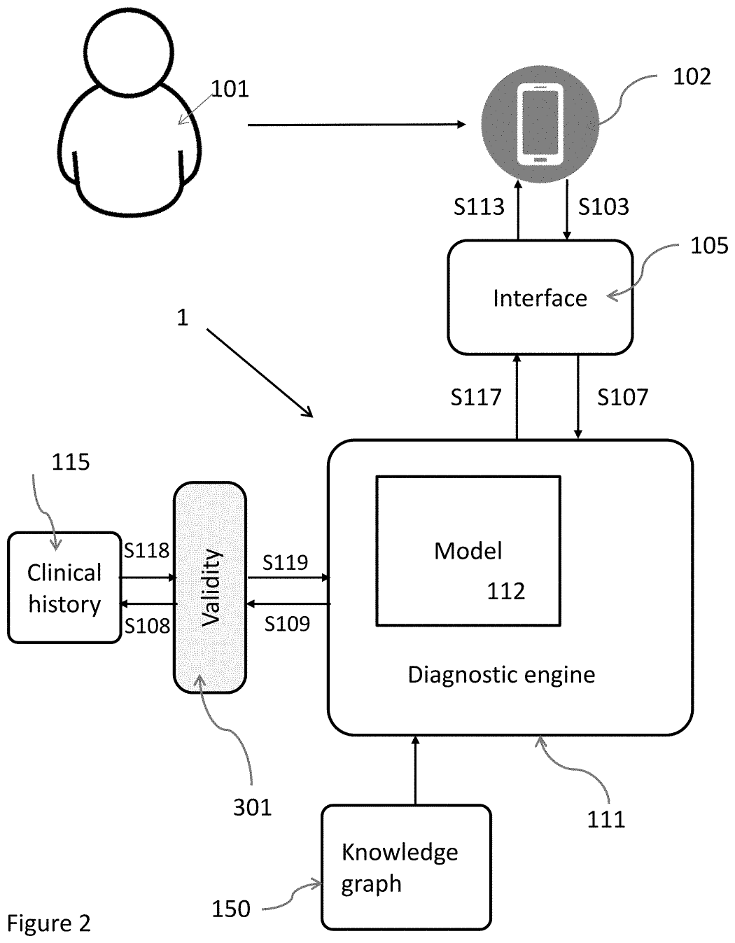

FIG. 2 is a schematic illustration of a medical diagnostic system 1 in accordance with an embodiment. For simplicity, the same reference numerals are used for components that have been described previously in relation to FIG. 1. Description of the features which have been described previously is omitted. Again, a patient 101 communicates with the system via a mobile phone 102, or any device which is capable of communicating information over a computer network, for example, a laptop, tablet computer, information point, fixed computer, etc. The user terminal 102 communicates through the interface 105 as described previously in relation to FIG. 1.

To begin the diagnosis process, the patient 101 inputs at least one symptom in step S103 via interface 105. The patient may also input one or more risk factors (for example, whether they are a smoker, their weight, etc.) and/or one or more diseases (for example, whether they are diabetic or asthmatic) and/or information relating to other items of medical data. The interface 105 may be adapted to ask the patient 101 specific questions. Alternately, the patient may simply enter free text.

This information is then sent to the diagnostic engine in step S107 in the same manner described previously. As described previously, the diagnostic engine 111 may extract a list of concepts corresponding to symptoms which are present in the user input, for example. This may correspond to a list of identifiers. The diagnostic engine may also extract concepts corresponding to diseases and/or risk factors and/or other items of medical information, which are present. It may also extract symptoms, diseases, and/or risk factors, or other items of medical data, which are used in the model and are identified by the user as absent.

In this embodiment, the diagnosis engine 111 is configured to identify the items of medical data, in this case symptoms, risk factors, and/or diseases, by their IRIs. Other types of identifiers may be used to represent these concepts; however, for simplicity, in the remainder of this specification we will refer to identifiers as IRIs.

The diagnostic engine 111 extracts the medical evidence (in this case at least one symptom which is identified as present, and optionally diseases and/or risk factors, and optionally those identified as absent) from the patient input. The diagnosis engine 111 comprises a probabilistic model 112 from which the probability of one or more diseases being present can be calculated, given the medical evidence provided, as has been described previously. From the calculated probabilities, the diagnosis engine 111 derives a likelihood that a disease is present and generates a diagnosis. The diagnosis may be output on the user terminal via steps S117 and S113. For example, for a given set of medical evidence (e.g., symptoms, diseases, or risk factors identified as present), if the diagnosis engine 111 calculates that P(disease=flu)=99%; P(disease=meningitis)=0.1%; and P(disease=measles)=0.2%, the diagnosis engine 111 will output that the disease is likely to be flu. The diagnosis may alternatively be output on another display device.

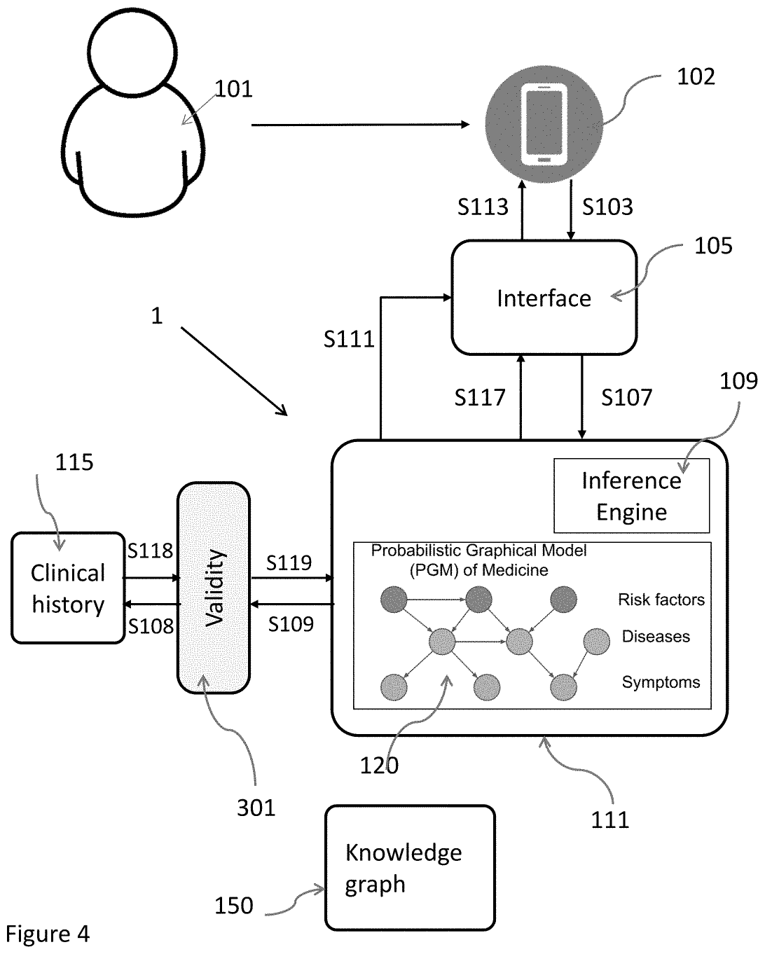

In an embodiment, the diagnosis engine 111 comprises an inference engine 109 and a PGM 120 as will be described in relation to FIG. 4 below. Other models may be used to perform the medical diagnosis, however.

The probabilistic model 112 also takes as input valid information (e.g., validated information) relating to items of medical data (such as symptoms, risk factors, and/or diseases) from a stored set of information relating to items of medical data (such as symptoms, risk factors, and/or diseases) associated with the user. The stored set of information relating to items of medical data is stored in the clinical history 115.

In the following example, the items of medical data are symptoms, risk factors, and/or diseases. However, other items of medical data, such as physiological data, recommendations, or behaviours may also be included. Physiological data may include height, weight, body mass index (BMI), or VO2max, for example. Recommendation may include medical advice such as “Do more exercise” or “Eat more vegetables.” Behaviour may include user behaviours such as “Physically active,” “Low physical activity,” or “Healthy eater.”

The diagnostic engine 111 first identifies the user corresponding to the received input. For example, the initial input from the user may comprise an HTTP request that contains a user ID and the text (comprising the at least one input symptom).

The diagnostic engine calls for the set of the symptoms, risk factors, and/or diseases which are to be used in the probabilistic model. The diagnostic engine sends this information to the validity module 301, together with the information identifying the user. The set of the symptoms, risk factors, and/or diseases may comprise a list of IRIs corresponding to the concepts in the PGM, for example. The validity module 301 then obtains the information relating to the user from the clinical history 112, based on the user identification.

This information from the clinical history may comprise one or more entries. Each entry may comprise an index, a patient ID, event information, the timestamp of the event, and the source of the event, for example. The event information may comprise a concept which was identified as present or absent in the event. The events may include items of medical data from notes by a human doctor, prescriptions, user reported symptoms or events, health checks, lab tests, healthcare providers' databases, or medical data from other sources. Events may be, for example, a diagnosis by the diagnostic engine or by a human doctor, a medical prescription, or an entry by a health monitoring service. The event information may further include information indicating whether the concept was identified as present (e.g., just the concept) or absent (e.g., the concept in combination with the NOT operator).

The validity module 301 extracts concepts from the events in the clinical history. In one example, the validity module 301 assigns 1, −1, or 0 to the extracted concept to indicate whether the concept is validly present (1), validly absent (−1), or this is unknown (0), and outputs a list of concepts, each with an assigned value of 1, −1, or 0. In another example, the validity module outputs a list of concepts which are identified as validly present in the clinical history. In yet another example, the validity module 301 may output a list of concepts together with a confidence level, C, where −1≤C≤1. The closer to “1” the value of C is, the higher the likelihood that a concept is validly present; and the closer to “−1” the value of C is, the higher the likelihood that a concept is validly absent.

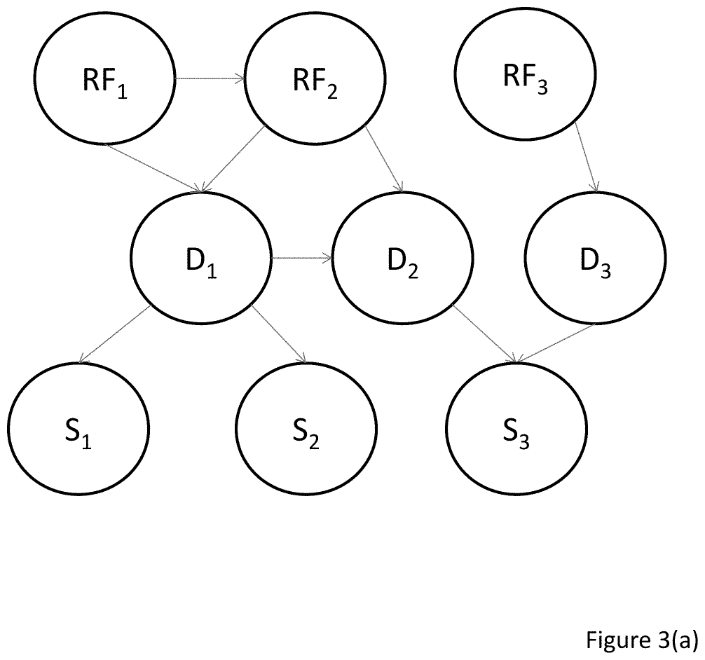

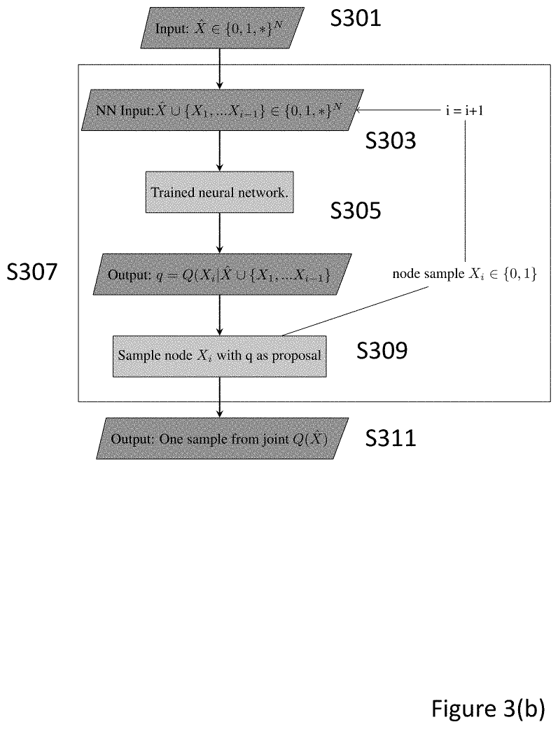

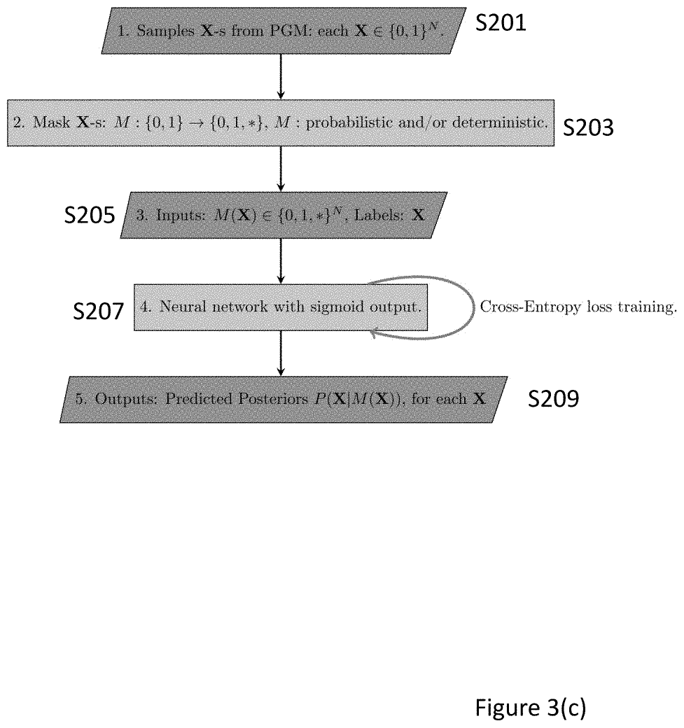

At the diagnostic engine, C may be compared to one or more threshold values in order to generate an input to the model. If a concept has a value greater than or equal to a first threshold value, it is considered to be present. If a concept has a value less than or equal to a second threshold value, it is considered to be absent. Otherwise, it is taken to be unknown. An example code that shows the manner in which the confidence level is used by the diagnostic engine to generate the model input is described further below in relation to FIG. 3(a).

The output of the validity module may comprise the information shown in the examples below. The output of the validity module comprises the valid information. The first example below shows a disease “Asthma” having a confidence=−1.

```
{
    “concept”: {
      “baseConcept”: {
        “label”: “Asthma”,
        “iri”:“https://protect-
eu.mimecast.com/s/9nIFC1wGKIM3E0MtkryYo?domain=bbl.health”
      },
      “modifiers”: [ ]
    },
    “validity”: {
      “confidence”: −1.0
    }
  },
```

Another example shown below shows a disease “Crohn's disease” having a confidence=0. In this example, a modifier with label “Chronic phase” is also defined.

```
  {
    “concept”: {
      “baseConcept”: {
        “label”: “Crohn's disease”,
        “iri”:“https://protect-
eu.mimecast.com/s/OS6rCpYkxSnmxJnSO2CbB?domain=bbl.health”
      },
      “modifiers”: [
        {
          “type”: {
            “label”: “HAS QUALIFIER”,
            “iri”: “https://protect-
eu.mimecast.com/s/Xe1oCqxlyu8Z7x8F0oDpX?domain=bbl.health”
          },
          “value”: {
            “label”: “Chronic phase”,
            “iri”:“https://protect-
eu.mimecast.com/s/X5NjCrkmzT8EDz8FMx_BZ?domain=bbl.health”
          }
        }
      ]
    },
    “validity”: {
      “confidence”: 0.0
    }
  }
```

A further example below shows a disease “Family history of malignant neoplasm of thyroid” having a confidence=1.

```
{
    “concept”: {
      “label”: “Family history of malignant neoplasm of thyroid”,
      “iri”:“https://protect-
eu.mimecast.com/s/A4KBC31yMipqRPpsg80AsX?domain=bbl.health”
    },
    “validity”: {
      “confidence”: 1.0
    }
  }
```

In the above examples, “confidence”: −1.0 represents validly “absent”; “confidence”: 0 represents “unknown”; and “confidence”: 1.0 represents validly “present.” Furthermore, in the above examples, the concepts are items of medical data, e.g., Asthma is an item of medical data, while the information about the medical data is the confidence, i.e., whether the concept is validly present, validly absent, or this is unknown. The values of −1.0, 0, and 1.0 are assigned to the concepts by the validity module 301. Example methods of assigning the confidence will be described in more detail below.

The validity module 301 takes as input the information from the clinical history 115 of the user, as described above, and extracts a set of symptoms, risk factors, and/or diseases to be used in the probabilistic model. The validity module 301 determines the information corresponding to each of the set of symptoms, risk factors, and/or diseases to be used in the probabilistic model from the stored information associated with the user (for example, whether the symptoms, risk factors, and/or diseases are validly present, validly absent, or this is unknown in this clinical history).

For example, the clinical history 115 of the user may comprise information from a doctor's note indicating that the patient has diabetes. The information about symptoms, risk factors, and/or diseases from the clinical history 115 of the user therefore comprises an indication that the disease “diabetes” is present. If this information is determined to be valid, the validity module may generate an entry “1” corresponding to the IRI representing the concept “diabetes.” The absence of a symptom, risk factor, and/or disease may be indicated by an entry of “−1,” for example. If it is unknown whether the symptom, risk factor and/or disease is present or absent, this may be indicated by a 0. As previously stated, other means of indicating this information may be used, however.

By comparing the symptoms, risk factors, and/or diseases from the diagnostic engine to the information about the symptoms, risk factors, and/or diseases from the clinical history, information about the symptoms, risk factors, and/or diseases used in the model is obtained (for example, whether each is validly present, or validly absent, or this is unknown).

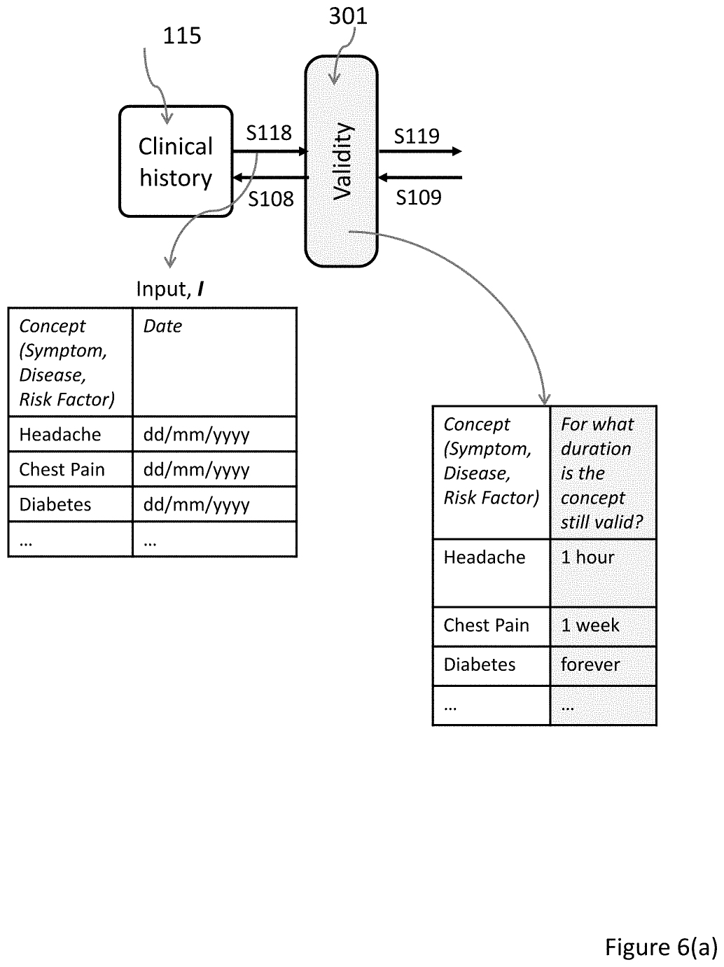

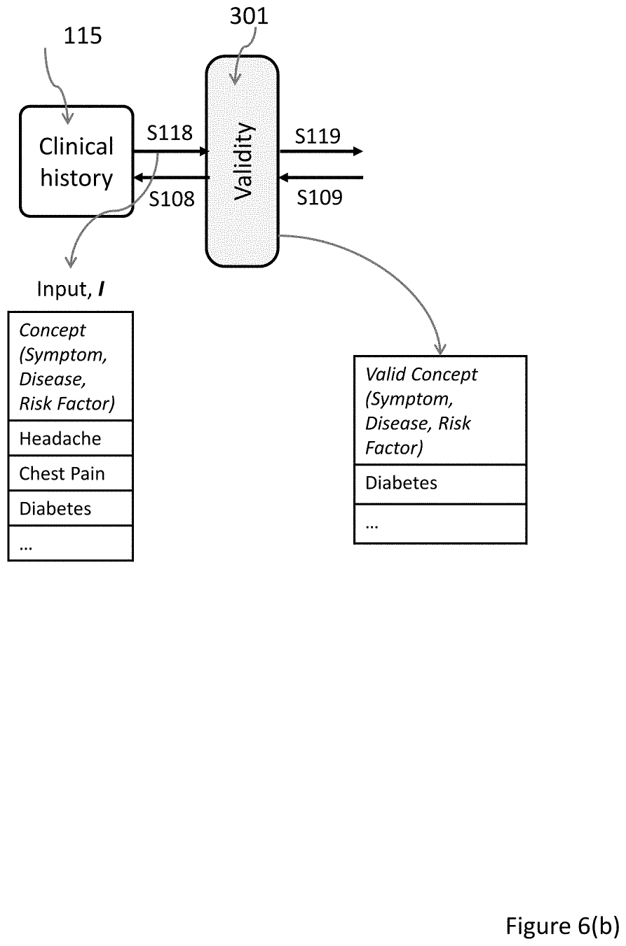

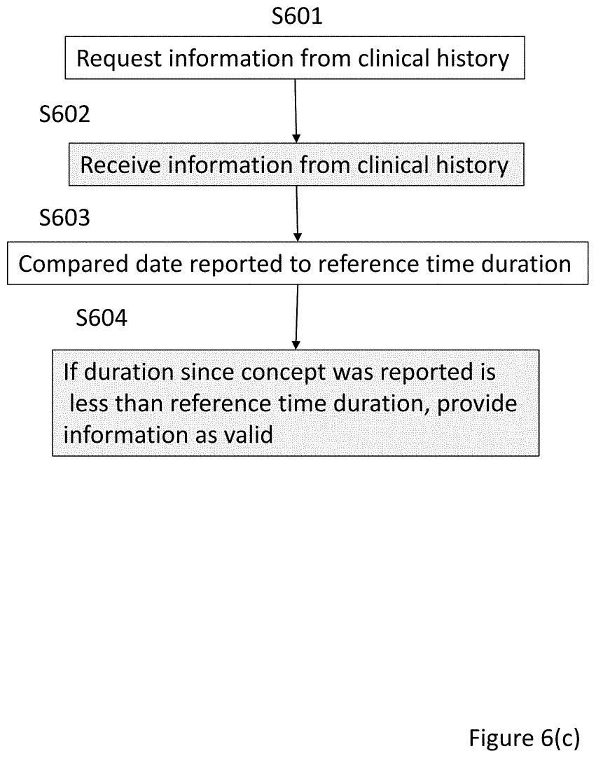

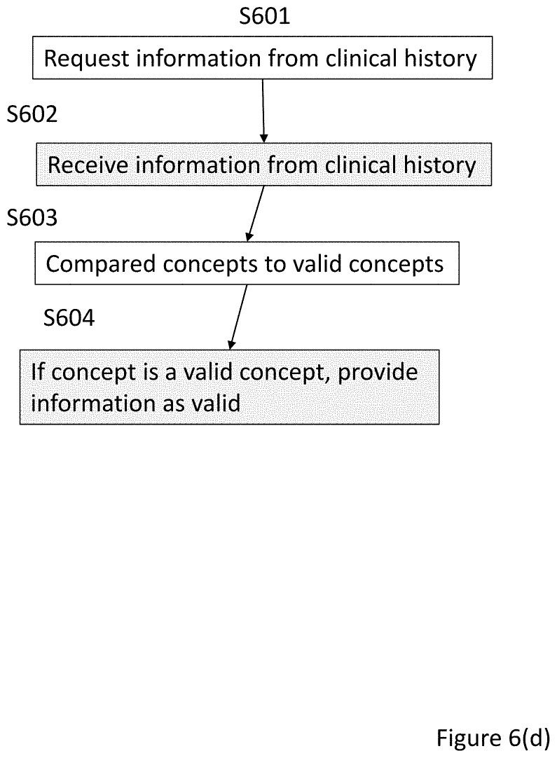

Thus, the validity module 301 first retrieves information relating to items of medical data from the stored clinical history 115, and then determines the validity of information for each item of medical data corresponding to the set of symptoms, risk factors, and/or diseases to be used in the probabilistic model. This may comprise determining which of the symptoms, risk factors, and/or diseases which are indicated as being present or absent in the clinical history 115 are validly indicated as such. Various methods of determining the validity are described below in relation to FIG. 6. The validity module 301 then passes valid information relating to the symptoms, risk factors, and/or diseases back to the diagnostic engine 111. This information is also taken as input to the probabilistic model 112 (as well as the information from the user input). The probabilistic model 112 determines the probability of the user having each of one or more diseases from the input information.

The diagnostic engine 111 is therefore configured to retrieve further information in step S109 from the stored clinical history, in addition to the information provided by the user in S107. The diagnostic engine 111 retrieves this information by calling a service called a “validity” service 301 (also referred to as the validity module). Various methods by which the validity module 301 determines if the information relating to a symptom, risk factor, and/or disease is valid in accordance with embodiments will be described in more detail below in relation to FIG. 6. Briefly, the validity module 301 acts as an interface between the diagnostic engine 111 and the patient's stored clinical history 115 that pre-processes information for items of medical data stored in the clinical history.

The validity module 301 may output the set of IRIs representing the symptoms, risk factors, and/or diseases used in the probabilistic model, together with the valid information (for example, the information may indicate, for each concept in the model, whether the concept is validly present, validly absent, or whether its presence/absence is unknown). This may be combined with the information received from the user and input into the model 112. The combination may be performed according to some pre-defined priority, as will be described below.

The validity module 301 receives information from the clinical history, comprising one or more concepts and information indicating that the concept is present or absent. The validity module 301 determines, for each concept corresponding to a symptom, risk factor, and/or disease used in the probabilistic model, whether the concept is validly present, validly absent, or whether its presence/absence is unknown. For example, if the validity module 301 determines from information in the clinical history that a symptom is present, the validity module 301 then determines whether this is valid. If this information is valid, the validity module 301 outputs to the diagnostic engine 111 that the symptom is present (1). If there is no information in the clinical history regarding the presence or absence of the symptom, or the information in the clinical history is determined to be not valid, the validity module 301 outputs to the diagnostic engine 111 that the presence/absence of the symptom is unknown (0). Similarly, if the clinical history provides information from which the validity module 301 determines that a symptom is absent, and this information is determined to be valid, the validity module 301 outputs to the diagnostic engine 111 that the symptom is absent (−1).

Data about the patient is held in the patient's clinical history 115. The clinical history comprises a record of information relating to items of medical data (i.e., symptoms, diseases, and/or risk factors) previously reported by the diagnosis engine 111. The clinical history may also include information relating to items of medical data from notes by a human doctor, prescriptions, user reported symptoms or events, health checks, lab tests, healthcare providers' databases, or medical data from other sources. In an embodiment, information indicating the source of each entry is also included with each entry. For example, information identifying whether the entry originated from a doctor, user, medical diagnosis system, or another source may be included.

The clinical history may contain entries over a large range of time, e.g., hours, days, weeks, months, or years. In an embodiment, the clinical history further comprises temporal information, corresponding to the reporting time of each entry. For example, each entry in the clinical history may comprise date information, indicating the day on which the entry was reported. For example, an entry in the clinical history may comprise an IRI representing a symptom, disease, or risk factor entered by a doctor at an appointment (for example, “backache”), and the date of the appointment. An entry previously reported by the diagnostic engine 111 may comprise a symptom input by the user together with the date on which the input occurred, and a second entry may comprise the disease determined by the diagnostic engine, together with the date on which the diagnosis occurred. Although date information is given here as an example, the temporal information may alternatively comprise time information, or only the year, for example. Date information may be stored so that the validity module 301 may determine validity based on the time since the concept was reported. However, validity may be determined using alternative criteria, and therefore, in some examples, the temporal information is not stored.

Items of medical data in the clinical history may comprise concepts, in particular the IRI(s) of the concept. The clinical history may also store information indicating whether the concept is present, absent, or unknown. For example, unary concepts (e.g., NOT headache) may be used to indicate that a concept is absent. Furthermore, the clinical history may include temporal information about concepts—that is, when they were reported. It may further comprise information about the source of the information (for example, doctor, patient, diagnosis system). The clinical history may store, for a particular patient: the Patient ID, event information comprising one or more concepts and information indicating whether each concept is present or absent, the date for each event, source for each event, and any extra application specific info (e.g., the confidence of a diagnosis or the free text the concepts were derived from).

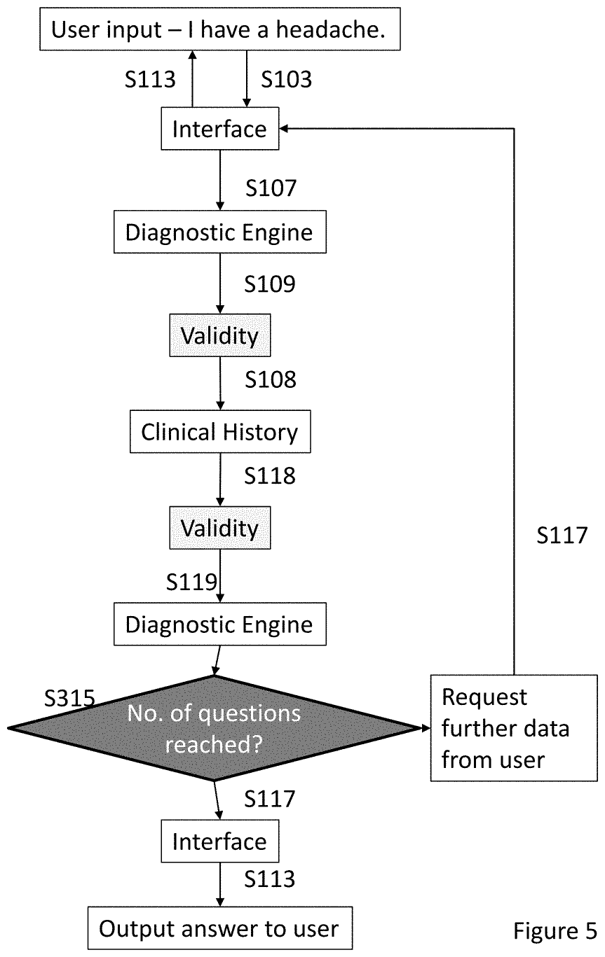

The probabilistic model 112 takes as input evidence, for example, the presence or absence of various symptoms, diseases and/or risk factors, from the clinical history and the information received from the user. For symptoms and risk factors where the patient has been unable to provide a response, and for which the status is unknown in the clinical history, these are initially assumed to be unknown. As will be described in relation to FIG. 5 below, the user and clinical history may only provide partial information, and therefore the system can be adapted to request further information from the patient. With this approach, several rounds of dialog between the user 101 and the diagnostic system 111 may be used to obtain information to make an accurate diagnosis. It is desirable that the questions asked of the user are relevant and thus lead to a more accurate diagnosis.

In an embodiment, some or all of the valid information obtained from the validity module is confirmed with the user before being input to the model. For example, some or all of the valid information may be sent to the user through the user interface, and the user requested to confirm the information is correct, and correct the information if incorrect. The confirmed and/or corrected information received from the user is then sent back to the diagnostic engine and the input evidence generated from the confirmed/corrected information.

Retrieving information from the clinical history 115 may provide additional evidence that was not reported by the user. The user clinical history is likely to comprise a diverse data set, however, with conflicting or erroneous data. Some of the information derived from the clinical history 115 may not be valid and may be conflicting. For example, the user may have reported a high temperature 3 years ago. One day ago, the user may have been asked if they have a high temperature and may report that they do not. Thus, the information from the clinical history may be conflicting. If invalid information is entered in the diagnosis engine 111 of FIG. 4, then the calculated probabilities may distort the likelihood of a particular disease and result in inaccurate diagnosis.

The data may therefore be pre-processed, before inputting into the model, so that only valid information is inputted. Validity may be determined based on temporal information. For example, the information indicating that the user has a high temperature was reported 3 years ago, and therefore likely to be invalid. Inputting the information indicating that the user has a high temperature to the probabilistic model may prevent the system from performing diagnosis. The system pre-processes the information from the stored information to identify a subset of valid information such that an accurate diagnosis can be obtained.

Further pre-processing to resolve any remaining conflicts in the data may be subsequently performed based on the information indicating the source of the data, for example, as will be described below.

FIG. 4 is a schematic illustration of a medical diagnostic system 1 in accordance with an embodiment. The diagnostic engine 111 comprises an inference engine 109 and a probabilistic graphical model (PGM) 120. Although an embodiment in which a PGM is used is described here, other models can alternatively or additionally be used, for example, one or more neural networks.

In the system shown in FIG. 4, and as described previously, follow-up questions may be asked by the interface 105. How this is achieved will be explained later. First, it will be assumed that there are no follow-up questions. This will be used to explain the basic procedure. However, a variation on the procedure will then be explained where the diagnosis engine, once completing the first analysis, requests further information.

Inference engine 109 performs Bayesian inference on PGM 120. PGM 120 will be described in more detail with reference to FIG. 3(a). In the medical diagnosis system, performing inference on the PGM provides the likelihood of a set of diseases, based on the evidence provided.

The inference engine 109 calculates “likelihood” (conditional marginal probability) P(Disease_i|Evidence) for all diseases.

In addition, the inference engine can also determine:

P(Symptom_i | Evidence),
P(Risk factor_i | Evidence).

From this, it can transmit back information in step S117 concerning the “likelihood” of a disease—in other words, a diagnosis—given the evidence supplied by the patient 101 and the clinical history. The interface 105 can then supply this information back to the mobile phone 102 of the patient in step S113.

Due to the size of the PGM 120, it may not be possible to perform exact inference using inference engine 109 in a realistic timescale. Instead, the inference engine 109 may perform approximate inference. The inference engine may be configured to perform approximate inference using importance sampling over conditional marginals. However, other methods may be used such as variational inference, other Monte Carlo methods, etc.

Approximate inference may comprise sampling from an independent ‘proposal’ distribution, which ideally is as close as possible to the target (true posterior distribution). The inference engine uses an approximation of the outputs of the probabilistic graphical model as proposals for subsequent sampling. One approach when applying Bayesian networks for medical decision-making is to use the model prior as the proposal distribution. Other approaches can be used, for example, generating a proposal distribution using a neural network. An example of this approach will be described below, however various other methods may be used.

Inference may be performed by considering the set of random variables, X={X₁, . . . X_{N}}. A BN is a combination of a directed acyclic graph (DAG), with Xᵢ as nodes, and a joint distribution of the Xᵢ, P. The nodes Xᵢ correspond to the risk factors, symptoms, and diseases as shown in FIG. 3(a), for example, and described in more detail below. The distribution P can factorize according to the structure of the DAG:

$$P ( {X}_{1} … , {X}_{n} ) = {\prod }_{i = 1}^{A} P ( {X}_{i} \vert P a ( {X}_{s} ) ) = P ( {X}_{1} ) {\prod }_{i = 2}^{N} P ( {X}_{i} \vert {X}_{1} , … , {X}_{i - 1} ) . \qquad ( 1 )$$

Where P(Xᵢ|Pa(Xᵢ)) is the conditional distribution of Xi given its parents, Pa(Xᵢ). The second equality holds as long as X₁; X₂; : : : , X_{N} are in topological order.

Now, a set of observed nodes is considered, [symbol] ⊂X and their observed values \hat{x}. To conduct Bayesian inference when provided with a set of unobserved variables, say [symbol] ⊂X\ [symbol] , the posterior marginal is computed:

$$P ( {X}_{𝒰} \vert {X}_{𝒪} = \hat{x} ) = \frac{P ( {X}_{𝒰} , {X}_{𝒪} = \hat{x} )}{P ( {X}_{𝒪} = \hat{x} )} = \frac{P ( {X}_{𝒰} ) P ( {X}_{𝒪} = \hat{x} \vert {X}_{𝒰} )}{P ( {X}_{𝒪} = \hat{x} )} \qquad ( 2 )$$

In the optimal scenario, Equation (2) could be computed exactly. However, as noted above, exact inference becomes intractable in large BNs as computational costs grow exponentially with effective clique size, in the worst case, becoming an NP-hard problem.

In an embodiment, importance sampling is used to perform approximate inference. Here, a function ƒ is considered for which its expectation, Ep[ƒ], is to be estimated, under some probability distribution P. It is often the case that we can evaluate P up to a normalizing constant, but sampling from it is costly.

In Importance Sampling, expectation Ep[ƒ] is estimated by introducing a distribution Q, known as the proposal distribution, which can both be sampled and evaluated. This gives:

$$\begin{matrix}{E}_{p} [ f ] = \int f ( x ) P ( x ) dx \\ = \int f ( x ) \frac{P ( x )}{Q ( x )} Q ( x ) dx \\ = {lim}_{n -> ∞} \frac{1}{n} {\sum }_{i = 1}^{n} f ( {x}_{i} ) {w}_{i} ,\end{matrix} \qquad ( 3 )$$

Where xᵢ˜Q and where wᵢ=P (xᵢ)/Q(xᵢ) are the importance sampling weights. If P can only be evaluated up to a constant, the weights need to be normalized by their sum.

In the case of inference on a BN, the strategy is to estimate P( [symbol] ) with an importance sampling estimator if there is appropriate Q to sample from. One approach when applying Bayesian networks for medical decision-making is to use the model prior as the proposal distribution Q.

Alternatively, a further model may be used to generate a proposal distribution corresponding to a joint distribution Q=P( [symbol] ). For example, the evidence may be passed to a universal marginaliser (UM). A UM is a neural network that has been trained to approximate the outputs of the PGM, for example, a single feedforward neural network, or a neural network which comprises several sub-networks (such that the whole architecture is a form of an auto-encoder-like model but with multiple branches). The UM returns probabilities to be used as proposals to the inference engine, based on the input evidence. The inference engine then performs importance sampling using the proposals from the UM as estimates.

An example inference method using a Universal Marginalizer has been described in the document Douglas, L., Zarov, I., Gourgoulias, K., Lucas, C., Hart, C., Baker, A., Sahani, M., Perov, Y. and Johri, S., 2017, A Universal Marginalizer for Amortized Inference in Generative Models, arXiv preprint arXiv:1711.00695, which is incorporated herein by reference.

The UM (e.g., a feedforward neural network) may be trained by sampling from the PGM. An example training process for the above described UM involves generating samples from the underlying BN, in each sample masking some of the nodes, and then training with the aim to learn a distribution over this data. An example training process using this approach is illustrated in FIG. 3(c).

The UM model is trained off-line by generating samples from the original BN (PGM) via ancestral sampling in step S201. In an embodiment, unbiased samples are generated from the probabilistic graphical model (PGM) using ancestral sampling. Each sample is a binary vector which will be the values for the classifier to learn to predict.

For the purpose of prediction, some nodes in the sample may then be hidden, or “masked” in step S203. This masking is either deterministic (in the sense of always masking certain nodes) or probabilistic over nodes. In an embodiment, each node is probabilistically masked (in an unbiased way), for each sample, by choosing a masking probability p˜U[0,1] and then masking all data in that sample with probability p.

The nodes which are masked (or unobserved when it comes to inference time) are represented consistently in the input tensor in step S205. Different representations of obscured nodes will be described later; for now, they will be represented as a ‘*’.

The neural network is then trained using a cross entropy loss function in step S207 in a multi-label classification setting to predict the state of all observed and unobserved nodes. The output of the neural net can be mapped to posterior probability estimates. Any reasonable, i.e., a twice-differentiable norm, loss function could be used. However, when the cross entropy loss is used, the output from the neural net is exactly the predicted probability distribution.

The trained neural network can then be used to obtain the desired probability estimates by directly taking the output of the sigmoid layer. This result could be used as a posterior estimate.

However, the output approximation can also be used as a proposal for any inference method (e.g., as a proposal for Monte Carlo methods or as a starting point for variational inference, etc.). An example method of importance sampling is described in relation to FIG. 3(b).

In the above discussion of Importance sampling, we saw that the optimal proposal distribution Q for the whole network is the posterior itself P( [symbol] ), and thus for each node the optimal proposal distribution is Qₒₚₜ=P(Xᵢ∈ [symbol] ∪X_{S}), where [symbol] are the evidence nodes and X_{S} are the already sampled nodes before sampling Xᵢ.

As it is now possible using the above UM to approximate, for all nodes, and for all evidences, the conditional marginal, the sampled nodes can be incorporated into the evidence to get an approximation for the posterior and use it is as proposal. For node i specifically, this optimal Q* is:

Q*ᵢ=P(Xᵢ|{X₁, . . . , Xᵢ₋₁}∪ [symbol] =\hat{x})≈UM({X₁, . . . Xᵢ₋₁}∪ [symbol] )i=Qᵢ (5)

The process for sampling from these approximately optimal proposals is illustrated in the algorithm 1 below and in FIG. 3(b) where the part within the box is repeated for each node in the BN in topological order.

In step S301, the input is received and passed to the UM (NN). The NN input is then provided to the NN (which is the UM) in step S303. The UM calculates in step S305, the output Q that it provides in step S307. This is provided to the Inference engine in step S309 to sample node Xᵢ from the PGM. Then, that node value is injected as an observation into \hat{x}, and it is repeated for the next node (hence ‘i:=i+1’). In step S311, we receive a sample from the approximate joint.

Algorithm 1 Sequential Universal Marginalizer importance sampling

| | |
|---|---|
| 1: | Order the nodes topologically X₁, . . . X_{N}, when N is the total number of nodes. |
| 2: | for j in [1, . . . , M] (where M is the total number of samples): do |
| 3: | \tilde{x}_{S} = ∅ |
| 4: | for i in [1, . . . N]: do |
| 5: | sample node xᵢ from Q(Xᵢ) = UM( [symbol] ) [symbol] ≈ P(X [symbol] P\|X_{S}, [symbol] ) |
| 6: | add xᵢ to \tilde{x}_{S} |
| 7: | [x_{S}]ⱼ = \tilde{x}_{S} |
| 8: | ${w}_{j} = {\prod }_{\text{?}}^{\text{?}} \frac{{P}_{i}}{{Q}_{i}} ( where {P}_{i} is the likelihood , {P}_{i} = P ( {X}_{i} = {x}_{i} \text{\textbar} {x}_{S ⋂ P \text{?} ( X \text{?} )} ) and {Q}_{i} = Q ( {X}_{i} = {x}_{i} ) )$ |
| 9: | ${E}_{p} [ X ] = \frac{{\sum }_{\text{?}}^{\text{?}} {X}_{\text{?}} {w}_{\text{?}}}{{\sum }_{\text{?}}^{\text{?}} {w}_{\text{?}}} ( as in standard IS )$ |

[symbol]

indicates data missing or illegible when filed

That is, following the requirement that parents are sampled before their children and adding any previously sampled nodes into the evidence for the next one, we are ultimately sampling from the approximation of the joint distribution. This can be seen by observing the product of the probabilities we are sampling from.

It can be seen that the proposal Q, constructed in such a way, becomes the posterior itself:

$$\begin{matrix}Q = {\sum }_{i = 1}^{N} {Q}_{i} & ( 6 ) \\ = {UM ( {X}_{𝒪} )}_{i} {\prod }_{i = 2}^{N} {UM ( {X}_{1} , … , {X}_{i - 1} , {X}_{𝒪} )}_{i} & ( 7 ) \\ \approx P ( {X}_{1} \vert {X}_{𝒪} ) {\prod }_{i = 2}^{N} P ( {X}_{i} \vert {X}_{1} , … , {X}_{i - 1} , {X}_{𝒪} ) & ( 8 ) \\ = P ( {X}_{1} , {X}_{2} , … , {X}_{n} \vert {X}_{𝒪} ) & ( 9 )\end{matrix}$$

This procedure requires that nodes are sampled sequentially, using the UM to provide a conditional probability estimate at each step. This can affect computation time, depending on the parallelization scheme used for sampling. However, parallelization efficiency can be recovered by increasing the number of samples, or batch size, for all steps.

In Importance Sampling, each node will be conditioned on nodes topologically before it. The training process may therefore be optimized by using a “sequential masking” process in the training process as in FIG. 3(c), where firstly we randomly select up to which node Xᵢ we will not mask anything, and then, as previously, mask some nodes starting from node Xᵢ₊₁ (where nodes to be masked are selected randomly, as explained before). This is to perform a more optimal way of getting training data.

Another approach might involve a hybrid approach as shown in Algorithm 2 below. There, an embodiment might include calculating the conditional marginal probabilities only once, given the evidence, and then constructing a proposal for each node Xᵢ as a mixture of those conditional marginals (with weight β) and the conditional prior distribution of a node (with weight (1−β)).

Algorithm 2 Hydrid UM-IS

| | |
|---|---|
| 1: | Order the nodes topologically X₁, . . . X_{N}, when N is the total number of nodes. |
| 2: | for j in [1, . . . , M] (where M is the total number of samples): do |
| 3: | \tilde{x}_{S} = ∅ |
| 4: | for i in [1, . . . N]: do |
| 5: | sample node xᵢ from Q(Xᵢ) = βUMᵢ( [symbol] ) + (1− β)P(Xᵢ = xᵢ\|x_{S∩P} [symbol] _{(X} [symbol] ₎) |
| 6: | add xᵢ to \tilde{x}_{S} |
| 7: | [x_{S}]ⱼ = \tilde{x}_{S} |
| 8: | ${w}_{j} = {\prod }_{\text{?}}^{\text{?}} \frac{{P}_{i}}{{Q}_{i}} ( where {P}_{i} is the likelihood , {P}_{i} = P ( {X}_{i} = {x}_{i} \text{\textbar} {x}_{S ⋂ P \text{?} ( X \text{?} )} ) and {Q}_{i} = Q ( {X}_{i} = {x}_{i} ) )$ |
| 9: | ${E}_{p} [ X ] = \frac{{\sum }_{\text{?}}^{\text{?}} {X}_{\text{?}} {w}_{\text{?}}}{{\sum }_{\text{?}}^{\text{?}} {w}_{\text{?}}} ( as in standard IS )$ |

[symbol]

indicates data missing or illegible when filed

While this hybrid approach might be easier and potentially less computationally expensive, in cases when P(Xᵢ|X_{S}∪ [symbol] ) is far from P(Xᵢ| [symbol] ), this will be just a first-order approximation, hence the variance will be higher and we generally need more samples to get a reliable estimate.

The intuition for approximating P(Xᵢ| [symbol] ∪X_{S}) by linearly combining P(Xᵢ|Pa(Xᵢ)) and UM( [symbol] )ᵢ is simply that UM( [symbol] )ᵢ will take into account the effect of the evidence on node i and P(Xᵢ|Pa(Xᵢ)) will take into account the effect of X_{S}, namely the parents. Note that β could also be allowed to be a function of the currently sampled state and the evidence, for example, if all the evidence is contained in parents, then=0 is optimal.

Although the above describes an inference method using importance sampling, alternatively, the sampling step may be omitted, and the trained UM can be used to directly approximate the posterior. The output of the UM comprises a vector of conditional marginal probabilities for every node in the BN, whether observed or not (if node Xᵢ is observed, the marginal posterior distribution for it will be trial, i.e., P(Xᵢ| [symbol] )=1 or P(Xᵢ| [symbol] )=0). The probabilities corresponding to the disease nodes can then be used directly for diagnosis, omitting the sampling step.

Although one method of performing inference has been described above, other models can be used to generate P(Disease_i|Evidence) for all diseases.

FIG. 3(a) is a depiction of a graphical model of the type used in the system of FIG. 4, according to an embodiment. The graphical model provides a natural framework for expressing probabilistic relationships between random variables, to facilitate causal modelling and decision making. In the model of FIG. 3(a), when applied to diagnosis, D stands for disease, S for symptom, and RF for Risk Factor. The model therefore has three layers: risk factors, diseases, and symptoms. Risk factors (with some probability) influence other risk factors and diseases, and diseases cause (again, with some probability) other diseases and symptoms. There are prior probabilities and conditional marginals that describe the “strength” (probability) of connections.

In this simplified specific example, in the first layer, there are three nodes S₁, S₂, and S₃, in the second layer there are three nodes D₁, D₂, and D₃, and in the third layer, there are three nodes RF₁, RF₂, and RF₃.

In the graphical model of FIG. 3(a), each arrow indicates a dependency. For example, D₁ depends on RF₁ and RF₂. D₂ depends on RF₂ and D₁. Further relationships are possible. In the graphical model shown, each node is only dependent on a node or nodes from a different layer. However, nodes may be dependent on other nodes within the same layer.

In an embodiment, the graphical model of FIG. 3(a) is a Bayesian Network (BN). The network represents a set of random variables and their conditional dependencies via a directed acyclic graph. Thus, in the network of FIG. 3(a), given full (or partial) evidence over symptoms S₁, S₂, and S₃ and risk factors RF₁, RF₂, and RF₃, the network can be used to represent the probabilities of various diseases D₁, D₂, and D₃.

In summary, the PGM 120 captures the probabilistic relationship between entities such as risk factors, diseases, and symptoms. Given a set of evidence, the inference engine 109 performs Bayesian inference from the PGM 120 and calculates a “likelihood” (conditional marginal probability) of a disease given a set of evidence for all diseases. Performing exact inference is computationally expensive and approximations are generally used to speed up the computation. Example methods have been described above for performing this calculation.

Information about the concepts (e.g., S₁, S₂, and S₃, RF₁, RF₂, and RF₃, and D₁, D₂, and D₃ in the example) is obtained from the user input in steps S103 and S107, and from the valid clinical history in S119. How the information from both sources is combined will be described later. This information corresponds to the nodes in the PGM, and is taken as the input evidence into the inference engine (i.e., the observed nodes).

The value of the state of the node reflects whether the concept represented by the node is true (1)—that is, it should have an impact on the calculation of probabilities—or false (0)—that is, it should not have any bearing on the calculation. It will be understood that the state of a node can have a value that is not restricted to 0 or 1.

As described above, the validity module may output information for each concept indicating whether it is present (1), absent (−1), or unknown (0). This information is combined with the user input. In an embodiment, the concepts which are unknown are then discarded, such that the input to the model consists of the presence (1) or absence (0) of concepts in the model. In another embodiment, an output of 1 from the validity module is mapped to “1,” an output of −1 from the validity module is mapped to “0” and an output of 0 from the validity module is mapped to “0.5.”

For example, the input “evidence_set” that is inputted to the model may be obtained from the output of the validity module according to the code below. A node corresponding to an item of medical data can be present, Evidence(node=node, state=PRESENT),” or absent, “Evidence(node=node, state=ABSENT).” In the example below, the state of the node is determined by comparing a confidence value from the validity module, “validity.confidence.” Examples of the confidence value from the validity module are shown previously as example outputs from the validity module. Since the PGM model is based on probabilities, then PRESENT becomes probability 1 and ABSENT becomes probability 0. All other nodes may be assigned a probability of 0.5, for example.

```
evidence_set = EvidenceSet( )
evidence_set.add_all(
  Evidence(node=node, state=PRESENT)
  for node, validity in node_to_validity.items( )
  if node.is_boolean
  if validity.confidence >= PRESENT_CONFIDENCE
)
evidence_set.add_all(
  Evidence(node=node, state=ABSENT)
  for node, validity in node_to_validity.items( )
  if node.is_boolean
  if validity.confidence <= ABSENT_CONFIDENCE
)
```

PRESENT_CONFIDENCE is a first threshold used to determine whether the state is present. In an embodiment, PRESENT_CONFIDENCE=1. ABSENT_CONFIDENCE is a second threshold used to determine whether the state is absent. In an embodiment, ABSENT_CONFIDENCE=−1. For values of the validity.confidence which do not meet either threshold, state=0.5 may be assigned, for example.

For example, the input evidence may comprise a vector \hat{x}, in which each entry corresponds to a node of the PGM. In the example described above, in which the inference engine uses importance sampling together with a universal marginaliser, the input evidence vector \hat{x} is provided as input to the universal marginaliser (comprising a neural network). The output from the universal marginaliser is then provided to the inference engine to sample node Xᵢ (corresponding to a disease) from the PGM. Then, that node value is injected as an observation into \hat{x}, and it is repeated for the next node. Finally, a sample is received from the approximate joint distribution.

Alternatively, the sampling step may be omitted, the input evidence vector \hat{x} provided as input to the trained neural network, the output of the neural network being directly used as the probability of each node corresponding to a disease.

As described above, the input medical data vector \hat{x} may comprise an entry corresponding to each node in the PGM, where the entry is a 1 if the symptom, risk factor, or disease is indicated as present (as determined from the information in the user input and the output from the validity module), and the entry is a 0 if the symptom, risk factor, or disease is absent (as determined from the information in the user input or in the output from the validity module). A value of 0.5 may be assigned where the presence/absence is unknown.

Where there is a conflict between the user input and the valid information, or where the valid information itself comprises a conflict, a pre-defined priority is used to select the information used. This will be described below.

Using information from the clinical history 115 may allow the presence (and optionally absence) of a greater number of nodes to be determined initially. Having values for more of the nodes enables the diseases to be computed with greater certainty (higher probability). In other words, entering information about a larger number of concepts, e.g., by having access to a valid clinical history, into the diagnosis engine 111, enables the diagnosis engine to determine diseases with more certainty (higher probability) and arrive at an accurate diagnosis.

However, if a large amount of information is entered but some of it is not valid, this may prevent diagnosis. For example, if an input RF3=1 is taken directly from the clinical history (omitting the validity module 301), but is not valid (for example, it was recorded a certain time ago and is no longer true), the diagnostic engine may provide an inaccurate diagnosis. The values of the nodes corresponding to different concepts (risk factors, disease, or symptom) taken as input to the probabilistic model 112 affect the values of the computed probabilities, and therefore the accuracy of the diagnosis. By pre-processing the information from the clinical history 115, before inputting it into the model 112, only valid information is input to the model 112.

In the example described above, according to an embodiment, the diagnostic engine 111 is configured to take information from the interface in S107, where the input is converted to a list of IRIs of the concepts reported as present by the user, and from the clinical history, via the validity module 301, where the valid information is provided. According to the above example, valid information from the clinical history are assigned values of {0, 1} in the diagnostic engine, which represent ‘absent’ and ‘present’.

FIG. 5 illustrates how the system of FIG. 2 can ask relevant follow-up questions to the patient. FIG. 5 illustrates a method of medical diagnosis in accordance with an embodiment, which may be performed on the system illustrated in FIG. 2, for example. In step S119, the system of FIG. 2 makes a diagnosis (for example, using the method explained above). The system then determines which further questions should be asked of the patient 101. In an embodiment, the next further questions to be asked are determined on the basis of questions that reduce the entropy of the system most effectively.

In the method illustrated in FIG. 5, the system has a pre-determined number of questions to ask the user. In S315, it is determined whether this number of questions has been reached. If not, the probabilities of the different diseases are considered and the most relevant question to be asked is determined (e.g., that which reduces the entropy of the system most).

Once the user supplies further information, then this is then passed back to the diagnosis engine 111 to update evidence to produce updated probabilities. The evidence vector is updated with the new information. Further iterations may be performed until the allowed number of questions is reached. At this point, a final diagnosis is output. Where the new evidence obtained from the user conflicts with the previous evidence, this may be resolved by implementing a rule which gives priority to the more recent information, for example. The input to the model then comprises the more recent information (e.g., that “headache” is present) instead of the previous information (e.g., that “headache” is absent).

In the above described example, the system has a pre-determined allowable number of questions. However, alternatively, the system may determine whether a diagnosis is accurate and then determine whether to ask further questions. In this case, the diagnosis engine 111 determines:

P(Disease_i | Evidence) for all diseases
P(Symptom_i | Evidence),
P(Risk factor_i | Evidence).

It is possible to use a value of information analysis (VoI) to determine from the above likelihoods whether asking a further question would improve the probability of diagnosis. For example, if the initial output of the system seems that there are 9 diseases each having a 10% likelihood based on the evidence, then asking a further question will allow a more precise and useful diagnosis to be made. Further iterations may be performed until a diagnosis is obtained with sufficient certainty.

The evidence may comprise a vector, where elements of the vector corresponds to one particular item of medical data, and the value of the elements is 0 or 1 (representing absent or present). Further elements in the vector corresponding to the other nodes in the model may be assigned a value of 0.5, for example. Each piece of medical data corresponds to a concept (symptom, risk factor, or disease) which is used in the model. The diagnostic engine may receive a first vector representing the user input, and a second vector representing the valid input from the clinical history. Elements of the first vector representing the user input may have values of ‘1’ for all symptoms, diseases, or risk factors positively identified by the user as present, and values of ‘0’ for all symptoms, diseases, or risk factors that are absent. A value of 0.5 may be assigned for elements where the presence/absence is unknown. Elements of the vector representing the clinical history may have values of ‘1’ for all symptoms, diseases, or risk factors that are validly present, values of ‘-1’ for all symptoms, diseases, or risk factors that are validly absent, and values of ‘0.5’ if they are unknown.

Both vectors may be of the same length, with each element denoting a particular concept according to the above. Each entry may correspond to a concept used in the model, for example. The information is combined in the manner described below. The data that is passed to the inference engine 109 and the PGM 120 of the diagnosis engine 111 is derived from both the user input in S107 and the valid clinical history in S119. The vectors representing the user input and the valid clinical history may have different values—intuitively, this is expected because, for example, the user is expected to report symptoms that he is currently experiencing, while the clinical history will provide other concepts such as risk factors, symptoms, and diseases previously reported.

In this example, the output from the validity module indicates present, absent, or unknown. Alternatively, the output from the validity module may simply indicate present or not present, for example.

Where both of the first vector and the second vector indicate that the concept is present, the concept is indicated as present (1) in the input evidence. Where one of the first vector and the second vector indicates that the concept is present, and the other indicates that it is unknown, the concept is indicated as present (1) in the input evidence. Where both of the first vector and the second vector indicate that the concept is unknown, the concept is indicated as unknown in the input evidence (0.5). Where both of the first vector and the second vector indicate that the concept is absent, or where one of the first vector indicates that the concept is absent and the other indicates that it is unknown, the concept is indicated as absent (0) in the input evidence. Where one of the first vector indicates that the concept is present, and the other indicates that it is absent, there is a conflict and this is resolved using a pre-defined priority.

In an embodiment, in order to combine the information from the clinical history and the user input, a set of rules is implemented to resolve any conflicts. The rules reflect the priority of the information source. For example, information from the clinical history that originates from a doctor is prioritised over information from the user. Information from the user is prioritised over information from the clinical history from any other source. In this manner, a single input vector may be generated. For example, if the user inputs that they do not have asthma, but the clinical history comprises information from a doctor indicating that they do have asthma, the information from the clinical history is taken and asthma is indicated as present. Additional or alternative rules may be implemented, for example, prioritising more recent information. The data may be stored together and read from a system wide event bus. The resolution of conflicts is achieved based on policies.

In an alternative embodiment, a conflict may be resolved by requesting further confirmation from the user.

Conflicts refer to the case where information indicating both presence and absence of the same concept is provided. Where one of the user input or clinical history indicates unknown in relation to the concept, there is no conflict, and the information from the other source is taken. Where both the user and input and clinical history indicate the same, again there is no conflict, and the information from either is taken. Conflicts may also occur within the valid clinical history information. Resolution of such conflicts is performed after validity is determined but before the combined input evidence is generated. It may be performed at the validity module, for example. Such internal resolution may be performed on the same or different pre-defined priorities. This will be described in more detail below.

The combined vector is then passed as input to the PGM for the calculation of the probabilities.

FIG. 6(a) is a schematic illustration of a system 301 in accordance with an embodiment for determining the validity of medical information. The system obtains information regarding items of medical data associated with a user and stored in a clinical history 115. It determines the validity of the information relating to some or all of the items, e.g., symptoms, risk factors, and/or diseases, and provides the valid information as output.

In the below described examples, the information corresponds to information indicating whether the concept is present or absent. However, the system may only use “present,” for example. Alternative information may be provided.

The validity system 301 obtains information from a stored clinical history 115, which comprises medical data about a patient. The clinical history 115 may be a database stored within the same system as the validity system 301, or it may be located remotely in a separate system. The clinical history comprises a record of information relating to items of medical data (i.e., symptoms, diseases, and/or risk factors). These may be those previously reported by a diagnosis engine or notes from a human doctor, prescriptions, user reported symptoms or events, or medical data from other sources, as described above, for example.

Each entry in the clinical history may further comprise temporal information, for example, date information indicating the day on which the entry was reported. It may additionally or alternatively comprise information about the source of the information (for example, doctor, patient, diagnosis system, or other).

The validity system 301 may obtain the information from the stored clinical history 115 in response to some input. This input may be a request for information relating to a set of symptoms, risk factors, and/or diseases comprised in a model used by a medical diagnosis system, as described in the example relating to FIG. 4 above. Alternatively, it may be simply a request for the valid clinical history from, e.g., a doctor or user.

FIG. 6(c) shows a flow chart illustrating a method of determining validity in accordance with an embodiment. In step S108, the validity module 301 requests information from the patient's clinical history 115. This corresponds to S601 in FIG. 6(c). The clinical history service provides information to the validity module in step S118. This corresponds to S602 in FIG. 6(c). The information provided to the validity module may comprise concepts. The information may indicate whether the concept is present (for example, by including the concept) or absent (for example, by including the operator “NOT” together with the concept). Alternatively, the information may simply indicate that the concept is present. For each entry, the information may further comprise temporal information (when they were reported). It may further comprise information about the source of the information (for example, doctor, patient, diagnosis system).

In S603, the validity module determines the validity of the information. In an embodiment, the validity module 301 is configured to determine whether information relating to a concept (for example, presence or absence of items of medical data such as symptoms, diseases, and/or risk factors) recorded in the past is still valid at present. The validity is determined based on the temporal information. The date on which the concept was reported is compared to a stored reference time duration in this example.

For example, suppose an event of a headache has been reported 3.3 months ago. It is unlikely that such an event is relevant to a diagnosis in the present, and therefore such information is determined not to be valid (i.e., the presence of a headache is determined to be invalid and, in the absence of any other information relating to a headache, whether a headache is present or absent is reported as unknown). In another example, suppose an event of a chest pain has been reported 3 days ago. It is likely that such an event is relevant to a diagnosis in the present, and therefore such information is determined to be valid (i.e., the presence of chest pain is determined to be valid). In yet another example, suppose a condition of ‘Diabetes’ is associated to a patient. It is almost certain that such an event is relevant to a diagnosis in the present, and therefore such information should be tagged as valid.

Temporal information relating to when the concept was reported is compared with information indicating how long information relating to the particular concept is valid, in order to determine whether the specific information is valid at the current time.

In this example, the validity module 301 comprises a stored list of items of medical data, encoded as concepts, and the relevant time durations for which they remain valid. The validity module 301 determines the time since the entry was reported by comparing the current date and the date on which the information was reported.

The validity module 301 compares the time since the entry is reported with stored information relating to each concept, indicating the duration of time for which information relating to that concept is valid. The stored information indicating the duration of the validity may be generated by human doctors, for example. If the time since the entry is reported is longer than the time for which information relating to that concept is valid, the information in the entry is determined to be invalid. The validity module 301 outputs “unknown” in relation to the concept (indicated by a 0). If the time since the entry is shorter than the time for which information relating to that concept is valid, the information in the entry is determined to be valid. The validity module 301 outputs the valid information in relation to the concept (for example, present, indicated by 1, or absent, indicated by −1).

The output from the validity module S119 may be provided to a diagnostic engine, for example, as has been described previously. Thus, in S604, the valid information is provided.

The validity of an input concept is determined based on the time when it was reported. The time between the concept being reported and the validity of the concept being determined is termed a “time duration.” A reference time duration is a time duration for which a concept is known to be valid. It may be a time duration for which a concept is known to be valid within a certain confidence interval (e.g., 90%), for example. The reference time duration may be generated by a human expert, for example. The method comprises: looking up reference time durations for which information about symptoms, diseases, and/or risk factors are valid from a table, for each symptom, disease, or risk factor, comparing the reference time durations to the time duration from when the symptom, disease, or risk factor was reported, and providing information relating to the symptom, disease, or risk factor obtained from the clinical history if the reference time duration is greater than or equal to the time duration from when information about the symptom, disease, or risk factor was reported.

The validity module thus comprises a database that contains a list of concepts and an associated reference duration, which indicates for how long a concept remains valid. The reference duration may indicate how long a concept remains valid within a certain level of certainty, e.g., 90%, after an incidence of the concept has been reported. The information in this database may be entered by human experts.

Although in the above example, whether the information is valid is determined in the same manner regardless of whether the information indicates that the concept is present or absent, in an embodiment, the validity may be determined differently for each case. Thus, it may first be determined whether the information indicates that the concept is present or absent. If present, a first criteria is applied in order to determine validity. If absent, a second criteria is applied. This is because the two cases may not be symmetrical, especially in the case of symptoms. This may mean using a shorter validity duration for absent information, for example. The duration for absence may be based on statistics on how likely a patient is to acquire the condition, for example. It may mean confirming the absence with the user.

The examples illustrate simple concepts such as headache, however, the validity module 301 may also operate on complex concepts (e.g., Severe Headache).

In the above described example, the validity module 301 matches the received concepts from the clinical history with the stored reference duration associated with the concept. In an embodiment, the validity module 301 is configured to predict the duration for which a concept remains valid based on the duration of a related concept, the related concept being either parent or a child.

In FIG. 6(a), a table storing validity duration information relating to a set of concepts (symptoms, risk factors, and/or diseases) is used to determine the validity of information. However, FIG. 6(b) illustrates an alternative validity system 301 in which a table storing a set of concepts for which the information indicating that the concept present is permanently valid is stored. For example, information indicating the presence of diabetes is likely to be relevant to a diagnosis forever. In this example, only information indicating the presence of a concept is returned from the validity module.

FIG. 6(d) shows a flow chart of a method of determining validity in accordance with an embodiment. In step S601, the validity module 301 requests information from the patient's clinical history 115. In this example, the clinical history may provide information including concepts which are present in the clinical history in S602. Temporal information is not provided. The validity module 301 compares the list of concepts from the clinical history with the list of valid concepts in S603 to determine validity. Only information relating to the valid concepts is output in S604.

Concepts for indicating permanence may also be stored in the knowledge base, and used by the validity module to determine which concepts are permanently valid. For example, permanence may be represented by the simple concept “Is Permanent.” One or more concepts used in the model may comprise a simple concept indicating permanence. The validity module may use this information to determine whether information provided from the clinical history is valid, instead of a stored table.

Conflicting data points may exist in the valid clinical history. In an embodiment, the validity module 301 reconciles differences between entries in the valid clinical history. For example, the clinical history may provide two entries, relating to the same concept, to the validity module. Both may be determined to be valid, but one may indicate that the concept is present and the other may indicate that the concept is absent. In this case, there is a conflict within the valid clinical history.

In an embodiment, a set of rules are implemented to resolve such conflicts. These are applied before the valid clinical history is combined with the user input to generate the input evidence. In an embodiment, the rules reflect the priority of the information source. For example, information that originates from a doctor is prioritised over information from the patient or from the medical diagnosis system. For example, if one valid entry in the clinical history originates from the user and indicates that they do not have asthma, and another valid entry in the clinical history comprises information from a doctor indicating that they do have asthma, the information from the doctor is taken and asthma is indicated as present.

Where one entry indicates unknown, the information from the other source is taken since no conflict exists. Where both sources indicate the same, the information from either is taken as, again, no conflict exists.

Other rules may additionally or alternatively be implemented, for example, a more recent entry may be taken as having priority. Different policies may be specified, for example, doctors may have priority over self-reports, or it can be specified to trust one particular source only.

As explained above, the clinical history comprises a record of items of medical data (i.e., symptoms, diseases, or risk factors) previously reported by the diagnosis engine 111. The clinical history may also include notes from a human doctor, prescriptions, user reported symptoms or events, or medical data from other sources. However, information recorded by the user over time may contradict the risk factors or diseases flagged by the human doctor. If conflicting concepts are passed to the diagnosis engine, the diagnosis may not be accurate. In this case, where valid information about the same concepts are conflicting, the validity module 301 may be configured to pass information that has been entered by a human doctor, rather than that logged in the clinical history by the user, for example.

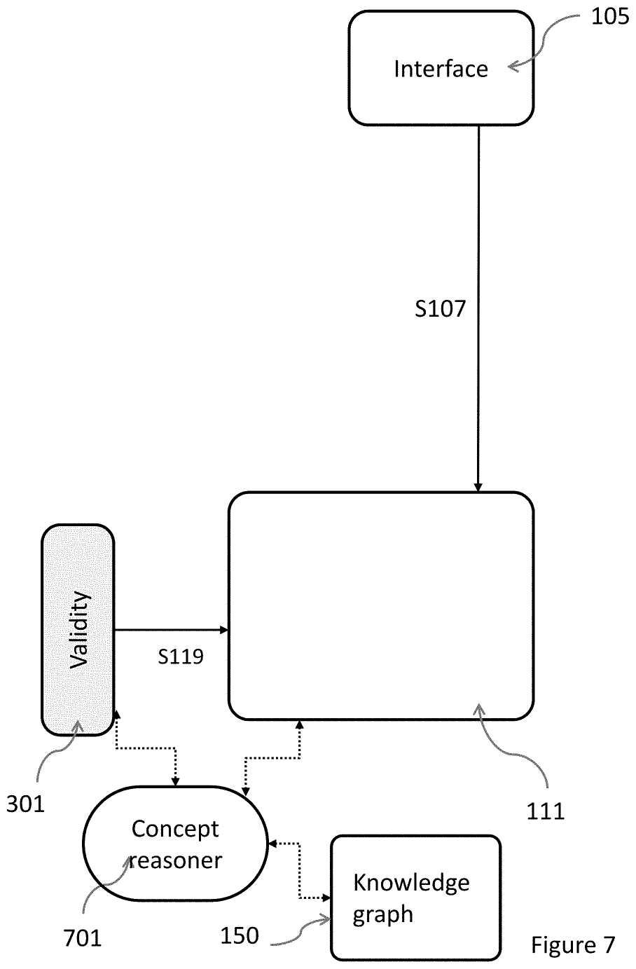

FIG. 7 shows part of a medical diagnosis system according to an embodiment, where a concept reasoner 701 is used.

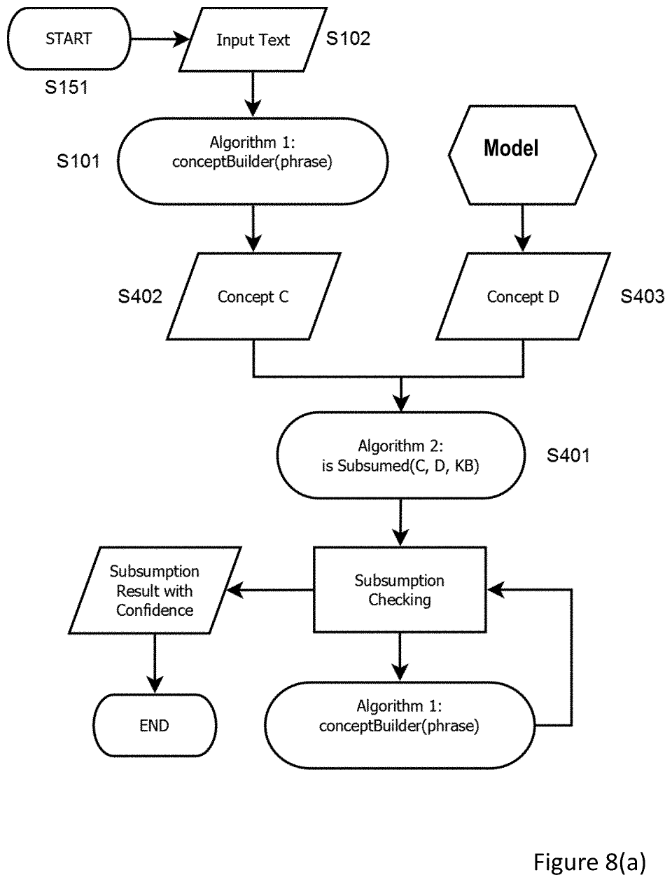

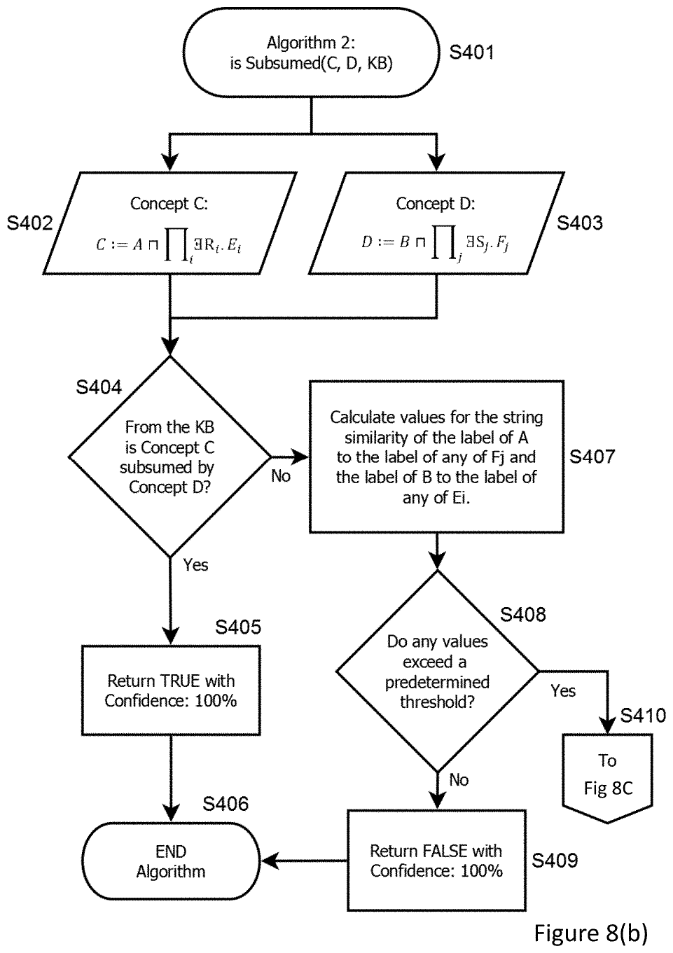

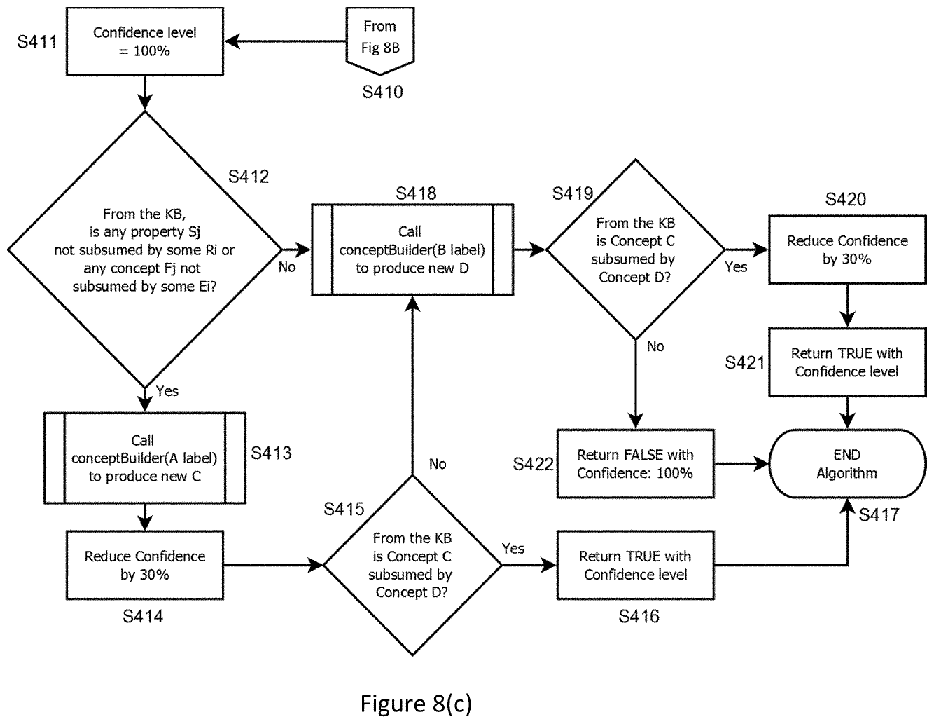

The concept reasoner 701 compares concepts (using subsumption). The concept reasoner 701 is used to compare the concepts used by the probabilistic model with those derived from the user input, in the manner shown in FIG. 8(a) (described below), for example. As has been described above, input text from a user is first processed using Natural Language Processing (NLP) techniques. The concept reasoner 701 then uses logic-based reasoning techniques to provide a concept comparison (using subsumption).

The NLP produces a query concept, which is generated using concepts and relations defined by the knowledge base. It is then checked if the query concept has a subsumption relationship with a candidate concept retrieved from the medical model (e.g., the PGM), and if no subsumption relationship is initially identified, the NLP process may be repeated with some optimisation. The query concept and the candidate concept comprise at least one elementary concept.

FIG. 8(a) shows a method of processing the user input using a concept reasoner, which may be used in a method of medical diagnosis in accordance with an embodiment.

NLP techniques are performed by algorithm 1 on the input text information to extract information relating to concepts. For example, in this step, a dependency tree may be generated with nodes representing the individual words from the input text, and optimised based on the linguistic relations between the nodes to merge and delete nodes. The output is defined in terms of concepts. For example, this step takes an input phrase and then calls an NLP dependency parser to tokenize the phrase and build its dependency tree. Various publically available parsers can be used for some of these steps. The parser may generate PGM concepts, for example (the parser is configured to “give the closest concept included in the list of PGM concepts”).

As has been described above, a list of concepts (the set of the symptoms, risk factors, and/or diseases) to be used in the probabilistic model are then called. In the example illustrated in FIG. 8(a), concept C is extracted from the user input and a list of concepts, including concept D, are returned as being used in the probabilistic model. For each concept returned as being used in the probabilistic model, algorithm 2 is performed. Algorithm 2 is described below.

In the below description, the symbol II represents logical conjunction. It is called AND for short. It can be used to form the conjunction of two concepts and create a new one. The conjunction of two concepts is interpreted as the intersection of the sets to which the two concepts are interpreted. For example: Professor II Male, which represents the notion of a male professor. As a whole, it is a concept. It is interpreted as the intersection of the sets to which concepts Professor and Male are interpreted.

The symbol ∃ is defined as the existential operator. It is called EXISTS for short. It can be used with a role and possibly combined also with a concept to form a new concept. For example: ∃hasChild represents the set of all things that have some child. Also: ∃hasChild.Male represents the set of all things that have a child, where the child is male.

The symbol [symbol] means “entails.” It is used to denote that something follows logically (using deductive reasoning) from something else. For example: ∃hasChild.Male [symbol] ∃hasChild since, if someone has a child which is male, then it follows that they necessarily have some child.

The symbol [symbol] is defined as the subclass operator (or the inclusion operator). It denotes a subclass relationship between two concepts. If one concept C is a subclass of another concept D, then the set to which C is interpreted must be a subset of the set to which D is interpreted. It can be used to form axioms. Intuitively it can be read as IF—THEN. For example: Male [symbol] Person can be read as “If something is a male then it is also a person.”

The symbol [symbol] has the standard set theoretic meaning of a subset relation between sets. The difference between the symbol [symbol] and [symbol] is that the latter denotes inclusion relation between classes. Classes are abstractions of sets. They don't have a specific meaning, but meaning is assigned to them via interpretations. So, when Male is written as a class, it acts as a placeholder for some set of objects. Hence, Male [symbol] Person means that every set to which Male is interpreted is a subset of every set that Person is interpreted. This relation is written as:

Male^{J} [symbol] Persons^{J}

where J is called an interpretation and it is a function that maps classes to sets.

Hence, Male^{J} is a specific set of objects.

SNOMED (Systematized Nomencalture of Medicine) is a systematically organised computer-processable collection of medical terms providing codes, terms, synonyms, and definitions, which may be used in clinical documentation. This information may be included in the knowledge base, for example.

Algorithm 2—Reasoning with Textual Knowledge

Based on a concept builder (Algorithm 1), a subsumption checking algorithm is presented that given two concepts, and can exploit both the ontological axioms as well as their textual information in order to compare them.

In more detail as shown in FIGS. 8(b) and (c), given a candidate subsumption C [symbol] S402, S403, the algorithm first attempts to check if it is entailed using a standard OWL reasoner S404. If the reasoner replies positively then the subsumption is guaranteed to hold and the algorithm returns TRUE with a confidence of 1.0 (100%) S405. It is to be understood that any sufficiently high confidence may be used as an output to match the concepts or subsumption in question. It may be preferred that the confidence is any value from: 99%, 98%, 97%, 96%, 95%, 94%, 93%, 92%, 91%, 90%, 85%, 80%, 75%, or any value there between.

If the OWL reasoner returns false, then the algorithm proceeds to use a heuristic method via function S407, to check whether to proceed to using a concept builder on the labels of C and/or D.

### Example 1—Subsumption Checking

The knowledge base can be expressed using [symbol] concepts and axioms and, moreover, conceptBuilder produces [symbol] concepts, hence it is possible to use efficient OWL reasoners like ELK to check for entailments and compare concept with respect to subsumption. However, in an embodiment, the knowledge base is very large; further, the complexity of the inference rules that these systems internally use do not scale. Consequently, in an embodiment, the knowledge base is loaded to the GraphDB triple-store. GraphDB is one commercially available triple-store with good performance and scalability guarantees; however, other triple-stores also exist and can be used. GraphDB's efficiency stems from the fact that it implements a weak set of inference rules; consequently, the system will not accurately compare the concepts computed by the conceptBuilder function.

To overcome this issue, in an embodiment, method includes a lightweight but approximate OWL reasoner which implements some additional consequence-based reasoning functionality on top of GraphDB using SPARQL queries. The SPARQL queries that are described below permit the simulation of some of the inference that a complete [symbol] reasoner will perform over a knowledge base, but it performs this up to a certain level, hence avoiding the aforementioned scalability issues.

Consider a patient with a face injury represented in his record using the SNOMED concept FaceInjury. Assume now that after analysing user text in the chatbot, the concept Injury Π∃findingSite.HeadStructure has been produced. Subsequently, the concepts, FaceInjury, and Injury Π ∃findingSite.HeadStructure are compared with respect to subsumption; i.e., it is required to check: FaceInjury [symbol] Injury Π ∃findingSite.HeadStructure.

SNOMED contains the axioms FaceInjury [symbol] ∃findingSite.FaceStructure and FaceStructure [symbol] HeadStructure, hence the subsumption relation between these two concepts holds. A system like ELK is able to answer positively about the entailment of this subsumption relation, however, GraphDB will not since its internal set of inference rules cannot handle concepts of the form ∃findingSite.FaceStructure. This example demonstrates an entailment that triple-stores cannot identify.

In order to provide some level of expressive reasoning, the method of this embodiment makes use of “concept expansion” prior to performing the subsumption check S404. The concept expansion is omitted from the Figures for the sake of readability. This approach is defined in detail below.

Let concepts C and D be defined as follows:

Definition 1:
C:=AΠΠᵢ₌₁^{m}∃Rᵢ.Eᵢ
D:=BΠΠⱼ₌₁ⁿ∃Sⱼ.Fⱼ

And let Function expandCon(C) be defined as

$${C}_{e x} : = A Π {\prod }_{i = 1}^{m} ∃ {R}_{i} \cdot {E}_{i} {\prod }_{k = m + 1}^{q} ∃ {r}_{k} \cdot {filler}_{k}$$

where each rₖ and fillerₖ is taken by the results of the following SPARQL query over some KB:
select ?r ?filler where {: C₁?r ?filler. ?r a owl:ObjectProperty.}

This query will search the knowledge base for axioms which match the format of C₁ [relation] [concept]. As mentioned above, SNOMED contains the axiom FaceInjury [symbol] ∃findingSite.FaceStructure. Since this axiom contains C (FaceInjury), a relation (findingSite) and a further concept (FaceStructure), this axiom will be one of the results returned by the SPARQL query. Cₑₓ in this example could therefore be defined as:

Cₑₓ=FaceInjury Π∃findingSite.FaceStructure

By using “concept expansion,” the algorithm can provide a greater level of expressive reasoning than is available through a typical triple-store deterministic reasoner while not requiring the demanding computational resources required by traditional expressive reasoning algorithms.

Then, subsumption between the concepts can be identified using the following function: Function isSubSet(C; D) returns true if the following tests hold, otherwise it returns false:

| | |
|---|---|
| 1. | query ask where {: A rdfs:subClassOf : B .} returns true. |
| 2. | for every jϵ [1; n] there exists iϵ [1; q] s.t. the following query |

returns true:
ask where {: Rᵢ rdfs:subClassOf : Sⱼ .: Eᵢ rdfs:subClassOf : Fⱼ .}

This query first whether that part A of concept C is subsumed by part B of concept D.

The query also checks each property Rᵢ to see if it is subsumed by any property Sⱼ and checks each concept Eᵢ to see if it subsumed by any concept Fⱼ. This can be an indirect subsumption and still hold true, e.g., A [symbol] X [symbol] B [symbol] A [symbol] B. Provided that all of these conditions are satisfied (i.e., A [symbol] B, each Rᵢ subsumed by some Sⱼ, each Eᵢ subsumed by some Fⱼ), then the query will return true and there it can be concluded that there is a subsumption relationship.

### Example 2—String Similarity

In some embodiments, conceptBuilder should not be used. For example, if the subsumption FootPain [symbol] HeadInjury is being tested then it is intuitively clear that there is no need to proceed into analysing the text of either of these concepts and using conceptBuilder bears the danger of leading to a false positive subsumption.

In order for the computer-implemented method to consider whether it should proceed with using conceptBuilder, using the concepts from definition 1 above, the function proceedToBuilder (line 10 of algorithm 2) S407 returns TRUE when applied on C and D if the following condition is satisfied:

For A (resp. B) there exists some ∃Sⱼ.Fⱼ (resp. ∃Rᵢ.Eᵢ) such that for [symbol] the label of A (resp. B) and for ε the label of Fⱼ (resp. Eᵢ) there exists sim(l,ε)≥T, where sim( ) is some string similarity algorithm and T is some threshold.

This can be interpreted as checking the similarity between the label of A with the labels of Fⱼ, and the label of B with the labels of Eᵢ S407, to see if any of the comparisons return a similarity score above a predetermined threshold S408.

For example, for the labels of the concepts in the (candidate) subsumption FootPain [symbol] RecentInjury, most string similarity measures like Levenshtein distance, Needleman-Wunch, and Smith-Waterman return a similarity score between 0.09 and 0.5 (a low degree). The threshold will not be exceeded and so the similarity of the two concepts being compared is too low. As such, the risk of generating a false-positive from the use of conceptBuilder is too high to proceed. Instead the algorithm returns FALSE and a confidence of 1.0 (100%) or thereabouts (S409).

In contrast, for the case of the concepts from Example 1 comparing the label of RecentInjury with that of concept Recently using the above three string similarity metrics returns a similarity score between 0.54 and 0.75 (a high degree) due to the conjunct ∃temporalContext.Recently. In that case, the algorithm would proceed to steps that would make use of the conceptBuilder (S411 on FIG. 4B).

### Example 3—analyseSubsumeesLabel( )

The next step of Algorithm 2 is to determine which concepts, either C or D, to apply the conceptbuilder to. This check is performed by the function analyseSubsumeesLabel_{KB}( ) (line 16, S413).

analyseSubsumeesLabel( ) works by checking if the following condition is satisfied based on concepts C and D as defined above, when provided with a knowledge base:

result = true if for every jϵ[1; n] there exists iϵ [1; q] s.t. the
following query returns true:
ask where {: Rᵢ rdfs:subClassOf : Sⱼ .: Eᵢ rdfs:subClassOf : Fⱼ .}
return !result

This query is the same test used in point 2 of the isSubSet( ) function, except that when the query returns true, the function returns false and vice versa. It should be noted that if the isSubSet( ) query above fails on point 1 (i.e., A is not subsumed by B) but point 2 holds true (there is subsumption between the other concepts of C and D), then conceptBuilder( ) should be performed on the label B from concept D instead of label A from concept C, as it is more likely that expanding concept B will lead to a positive subsumption result.

Considering again the two concepts C₁ and C₂ from Example 1:

C₁ := RecentInjury Π ∃findingSite.Head
C₂ : := Injury Π ∃findingSite.Head Π ∃temporalContext.Recently
∃temporalContext.Recently is not subsumed by any conjunct of similar
form in C₁.

Even if expandCon( ) is applied on C₁, no conjunct is added. Hence, analyseSubsumeesLabel(C₁,C₂) would return TRUE and proceed to use the conceptBuilder on the label of RecentInjury (line 17, S414).

In contrast, analyseSubsumeesLabel(C₂,C₁) returns FALSE, as for all of the conjuncts of the form ∃R.E in C₁, there is a conjunct of the same form in C₂ that subsumes it, i.e., ∃findingSite.Head is present in both C₁ and C₂. As a result, the algorithm instead skips lines 17-23 and instead runs the concept builder on the label of Injury (line 24, S418).

### Example 4—Further Use of conceptBuilder( )

Presuming that analyseSubsumeesLabel( ) returns TRUE (line 16, S412) and the conceptBuilder is applied to Concept C (line 17, S413), the algorithm is tracking a confidence level which is initialised at 100% (line 14, S411). After conceptBuilder has generated new concept C, the confidence level is reduced to a lower amount, e.g., it is reduced by 30% (line 18, S414). This reflects the concern that using the conceptBuilder increases the risk of a false positive result.

The subsumption between the revised concept C and the concept D is then checked (line 19, S415). If the subsumption is now true, the algorithm returns TRUE with the current confidence level (e.g., 70%) (line 20, S416).

If, however, the subsumption is still not true, then conceptBuilder is applied to Concept D (line 24, S418).

The subsumption between the revised concept C and the revised concept D is then checked (line 25, S419). If the subsumption is now true, the confidence level is further reduced (e.g., by a further 30%)(S420). The further use of conceptBuilder leads to an even greater risk of false positive; hence, a low confidence level is given. The algorithm then returns TRUE with current confidence level (e.g., 40%) (line 26, S421).

If, however, the subsumption is still not true, then the algorithm returns FALSE with 100% (or thereabouts) confidence level (line 29, S422). As the algorithm is unable to resolve any subsumption after the application of conceptBuilder to both concept C and D, it is assumed that there is no subsumption of C by D.

Returning to the analyseSubsumeesLabel( ) check (line 16, S412). if this function instead returns FALSE, then it is not appropriate to apply conceptBuilder to concept C (lines 17-24 are skipped) and instead, since it has already been determined that concept C is not subsumed by D (line 6, S404), conceptBuilder is applied only to D (line 24, S418). Once conceptBuilder is applied to concept D, the confidence level is reduced (e.g., to 70%).

The subsumption of the concept C by the revised concept D is checked (line 25, S419). If the subsumption is now true, the confidence level is reduced (e.g., by 30%)(S420) for the same reasons as given above. The algorithm then returns TRUE with the current confidence level (e.g., 70%) (line 26, S421).

If, however, the subsumption is still not true, then the algorithm returns FALSE with 100% (or thereabouts) confidence level (line 29, S422). As the algorithm is unable to resolve any subsumption after determining that it is inappropriate to apply conceptBuilder to concept C and applying it only to concept D, it is assumed that there is no subsumption of C by D.

The end output of Algorithm 2 is a Boolean (TRUE or FALSE) result representing whether C is subsumed by D and an outputted value representing the confidence level in that subsumption result.

Algorithm 2 Pseudocode

```
isSubSumed_{KB}(C,D)
wherein C and D are concepts of the form:
C := A Π Πᵢ∃Rᵢ.Eᵢ and D := B Π Πⱼ∃Sⱼ.Fⱼ
if KB [symbol]     C [symbol]     D then //isSubSet( )
  return <true,1.0>
end if
if !proceedToBuilder_{KB}(C,D) then
  return <false,1.0>
end if
C_{br} := C
d := 1.0
if analyseSubsumeesLabel_{KB}(C,D) then
  C_{br} := conceptBuilder_{KB}(A.label) Π Πᵢ∃Rᵢ.Eᵢ
  d := d − 0.3
  if KB [symbol]     C_{br} [symbol]     D then
    return <true,d>
  end if
end if
D_{br} := conceptBuilder_{KB}(B.label) Π Πⱼ∃Sⱼ.Fⱼ
if KB [symbol]     C_{br} [symbol]     D_{br} then
  return <true, d − 0.3>
end if
return <false,1.0>
```

The above describes an example of how the concept reasoner 701 may be used to determine whether a concept from the user input is a lower-level object which is a member of the higher class represented by the concept from the probabilistic model. For example, the concept reasoner can reason that “frontal headache” input by the user is a child concept of the concept “headache,” used by the probabilistic model.

In the same way, the concept reasoner 701 may be used by the validity module to determine whether concepts from the clinical history are subsumed by concepts in the probabilistic model. In this case, the input text in S102 of FIG. 8(a) corresponds to that retrieved from the clinical history, rather than that input by a user. Thus, if a clinical note reported “frontal headache” and the PGM comprises the concept “headache,” the only way to return that “headache” to present is to know that “frontal headache” is subsumed by “headache.” This is what the concept reasoner returns.

In an embodiment, the validity module 301 additionally or alternatively uses the concept reasoner 701 to derive the validity of a concept (a parent) from the validity of a sub-concept (a child). As described above in relation to FIGS. 6(a) and (b), for example, the validity module may comprise stored information allowing determination of the validity for a set of specific concepts only. This information may be a table comprising reference durations for a set of concepts, or a table comprising valid concepts, as described above, for example.

By using the concept reasoner 701, the validity module may determine the validity of information for concepts for which it does not have reference time durations or a permanently valid concept, if the concept is determined to be subsumed by a concept for which a reference time duration or a permanently valid concept are held. Thus, the same process described in FIG. 8(b) may be performed, where the concepts in S402, including concept C, corresponds to the concepts for which there is stored validity information (and thus S151, S102, and S101 are omitted), and the concepts in 403 correspond to those retrieved from the clinical history, rather than those used in the probabilistic model. This can be used as a “credulous” inference mechanism. For example, to obtain an estimate for “frontal headache’ it may refer to the duration of “headache.”

For example, if concept C corresponds to ‘frontal headache’ (child), and this is valid after a time of 1 hour, but the clinical history returns concept D, headache, the validity module determines that ‘frontal headache’ is subsumed by ‘headache’ through the concept reasoner 701, and derives that ‘headache’ (parent) must also be valid after a time of 1 hour. This rule can be summarised as:
validity(X, V) IF tagged_validity(X, F) OR (validity(Y, F) AND subclass(X, Y)),
   where X and Y are concepts, and V and F represent time. ‘tagged_validity’ refers to a validity value that is derived from a reference probability distribution or reference time duration that has been checked by a human supervisor, for example.

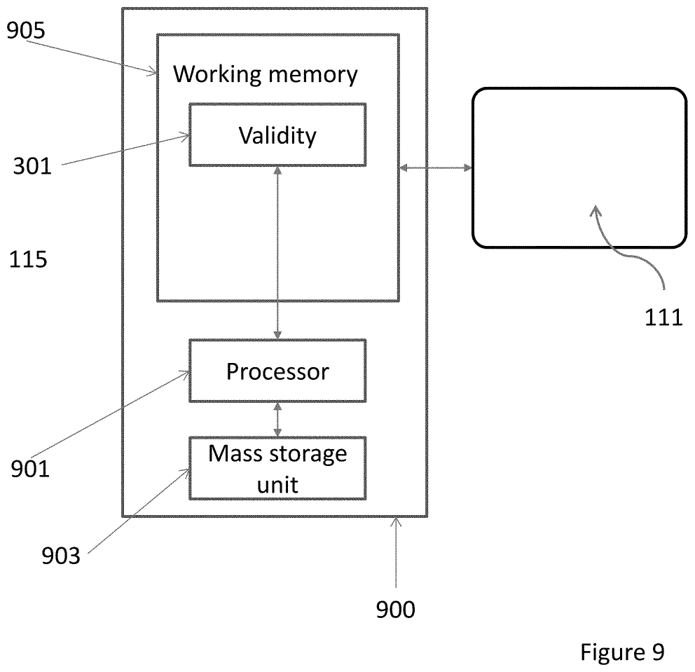

While it will be appreciated that the above embodiments are applicable to any computing system, an example computing system is illustrated in FIG. 9, which provides means capable of putting an embodiment, as described herein, into effect. As illustrated, the computing system 900 comprises a processor 901 coupled to a mass storage unit 903 and accessing a working memory 905. As illustrated, a validity module 301 is represented as a software product stored in working memory 905. Further functionality, such as the concept reasoner, may also be embodied as a software product stored in working memory 905. The clinical history may be stored in a Cassandra DB table, for example.

It will be appreciated that elements of the validity module 301 may, for convenience, be stored in the mass storage unit 903. It will also be appreciated that the computing system 900 is connected to and configured to communicate with other parts of the medical diagnosis system 1, such as the diagnosis engine 111. It will also be appreciated that the diagnosis engine 111 may be put into effect by means of a computing system similar to the system 900.

In use, the system receives data from a user. The programs, including the validity module, are then executed on the processor in the manner which is described with reference to the above figures. The processor may comprise logic circuitry that responds to and processes the program instructions.

While certain embodiments have been described, these embodiments have been presented by way of example only, and are not intended to limit the scope of the inventions. Indeed, the novel methods and apparatus described herein may be embodied in a variety of other forms; furthermore, various omissions, substitutions, and changes in the form of methods and apparatus described herein may be made.

1. A computer-implemented method for determining a validity of medical information, the method comprising:
obtaining, from a stored clinical history, information relating to a set of items of medical data associated with a user, the information comprising an indication of whether the item of medical data is present, wherein an item of medical data comprises a symptom, risk factor, disease, physiological data, recommendation or behaviour;
obtaining, from the stored clinical history, the source of the information indicating whether the item of medical data is present;
determining the validity of the information and providing valid information, wherein the validity is determined by comparing the item of medical data to a list of items of medical data which are permanently valid and determining that the information indicating that the item of medical data is present is valid if the item of medical data corresponds to an entry on the list, or by comparing a reference time duration to a time duration from when the information was reported in the stored clinical history and determining that the information is valid if the reference time duration is greater than the time duration from when the information was reported;
applying a policy to prioritise information based on the source information; and
identifying any conflicting valid information,
wherein any identified conflicts are resolved based on the priority.

2. (canceled)

3. The method according to claim 1, wherein when the source information indicates that the source is a human doctor, the information has priority.

4. (canceled)

5. The method according to claim 4, wherein there is a stored reference time duration associated with each of a plurality of items medical data.

6. The method according to claim 1, further comprising:
checking if an item of medical data from the set of items of medical data has a subsumption relationship with a candidate item of medical data for which information used to determine validity is stored.

7. The method according to claim 1, wherein the information comprises information indicating that the item of medical data is present or absent, and wherein for one or more of the items of medical data, there is a first reference time duration which is used when the information indicates that the item of medical data is present, and a second reference time duration which is used when the information indicates that the item of medical data is absent.

8. The method according to claim 1, wherein the valid information comprises a confidence level representing a likelihood that the corresponding item of medical data is absent or present.

9. The method according to claim 1, wherein the stored clinical history comprises information relating to items of medical data from healthcare providers' data sources.

10. The method according to claim 1, wherein the source information comprises information identifying whether the information comprising an indication of whether the item of medical data is present originated from one or more of: a doctor, a user, a medical diagnosis system, a healthcare providers' data sources, a prescription, a health check, a lab test.

11. A computer-implemented method for determining validity of medical information, the method comprising:
obtaining, from a stored clinical history, information relating to a set of items of medical data associated with a user, the information comprising an indication of whether the item of medical data is present, wherein an item of medical data comprises a symptom, risk factor, disease, physiological data, recommendation or behaviour;
obtaining, from the stored clinical history, the source of the information indicating whether the item of medical data is present;
determining the validity of the information and providing valid information, wherein the validity is determined by comparing the item of medical data to a list of items of medical data which are permanently valid and determining that the information indicating that the item of medical data is present is valid if the item of medical data corresponds to an entry on the list, or by comparing a reference time duration to a time duration from when the information was reported in the stored clinical history and determining that the information is valid if the reference time duration is greater than the time duration from when the information was reported;
identifying any conflicting valid information; and
resolving identified conflicts by excluding valid information based on the source information.

12. A system for determining validity of medical information, the system comprising:
an input configured to receive information relating to a set of items of medical data associated with a user from a stored clinical history, the information comprising an indication of whether the item of medical data is present, wherein an item of medical data comprises a symptom, risk factor, disease, physiological data, recommendation or behaviour, wherein the input is configured to receive, from the stored clinical history, the source of the information indicating whether the item of medical data is present;
a processor configured to determine the validity of the information, wherein the validity is determined by comparing the item of medical data to a list of items of medical data which are permanently valid and determining that the information indicating that the item of medical data is present is valid if the item of medical data corresponds to an entry on the list, or by comparing a reference time duration to a time duration from when the information was reported in the stored clinical history and determining that the information is valid if the reference time duration is greater than the time duration from when the information was reported, wherein the processor is configured to apply a policy to prioritise information based on the source information and identify any conflicting valid information, wherein any identified conflicts are resolved based on the priority; and
an output configured to provide valid information.

13. The system according to claim 12, wherein when the source information indicates that the source is a human doctor, the information has priority.

14. (canceled)

15. The system according to claim 14, wherein there is a stored reference time duration associated with each of a plurality of items medical data.

16. The system according to claim 12, wherein the information comprises information indicating that the item of medical data is present or absent, and wherein for one or more of the items of medical data, there is a first reference time duration which is used when the information indicates that the item of medical data is present, and a second reference time duration which is used when the information indicates that the item of medical data is absent.

17. The system according to claim 12, wherein the valid information comprises a confidence level representing a likelihood that the corresponding item of medical data is absent or present.

18. The system according to claim 12, wherein the stored clinical history comprises information relating to items of medical data from healthcare providers' data sources.

19. A system according to claim 12, wherein the source information comprises information identifying whether the information comprising an indication of whether the item of medical data is present originated from one or more of: a doctor, a user, a medical diagnosis system, a healthcare providers' data sources, a prescription, a health check, a lab test.

20. A system for determining validity of medical information, the system comprising:
an input configured to receive information relating to a set of items of medical data associated with a user from a stored clinical history, the information comprising an indication of whether the item of medical data is present, wherein an item of medical data comprises a symptom, risk factor, disease, physiological data, recommendation or behaviour, wherein the input is configured to receive, from the stored clinical history, the source of the information indicating whether the item of medical data is present;
a processor configured to determine the validity of the information, wherein the validity is determined by comparing the item of medical data to a list of medical data which are permanently valid and determining that the information indicating that the item of medical data is present is valid if the item of medical data corresponds to an entry on the list, or by comparing a reference time duration to a time duration from when the information was reported in the stored clinical history and determining that the information is valid if the reference time duration is greater than the time duration from when the information was reported, and wherein
the processor is configured to identify any conflicting valid information and resolve identified conflicts by excluding valid information based on the source information; and
an output configured to provide valid information.
